SEQUENCE LISTING (14 sequences)

SEQ ID NO: 1 - protein, 564 aa, synthetic construct
MDYKDDDDKVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRAAYSRLEDRRVRPTSSGDLYYIGTEQKLISEEDL

SEQ ID NO: 2 - protein, 539 aa, synthetic construct
MDYKDDDDKILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRAAYSRLEDRRVRPTSSGDLYYIGTEQKLISEEDL

SEQ ID NO: 3 - protein, 620 aa, synthetic construct
MDYKDDDDKPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTEQKLISEEDL

SEQ ID NO: 4 - protein, 702 aa, synthetic construct
MDYKDDDDKILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILGSSGASPAAPAPASPAAPAPSAPAGGMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGHMGSEQKLISEEDL

SEQ ID NO: 5 - protein, 817 aa, synthetic construct
MDYKDDDDKILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILGSSGASPAAPAPASPAAPAPSAPAGGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGHMGSEQKLISEEDL

SEQ ID NO: 6 - protein, 763 aa, synthetic construct
MDYKDDDDKHMGSMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGGSSGASPAAPAPASPAAPAPSAPAGGQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTEQKLISEEDL

SEQ ID NO: 7 - protein, 878 aa, synthetic construct
MDYKDDDDKHMGSMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSSGASPAAPAPASPAAPAPSAPAGGQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTEQKLISEEDL

SEQ ID NO: 8 - DNA, 1695 bp, synthetic construct
  source 1..1695: mol_type = other DNA
atggactataaagacgatgacgacaaagtggtgattctggataaacgttgttattgcaatctgctgattctgatcctgatgattagcgaatgtagcgttggtatcctgcactatgaaaaactgagcaaaattggtctggttaaaggtgtgacccgcaaatacaaaatcaaaagcaatccgctgaccaaggacatcgtgatcaaaatgattccgaatgtgagcaatatgagccagtgtaccggtagcgttatggaaaactataaaacccgtctgaatggtattctgaccccgattaaaggtgcactggaaatctataagaacaacacccatgatctggttggtgatgttcgtctggcaggcgttattatggctggtgttgcaattggtattgcaaccgcagcacagattaccgcaggcgttgcactgtatgaagcaatgaaaaatgccgacaacatcaacaaactgaaaagcagcattgaaagcaccaatgaagcagttgttaaactgcaagaaaccgcagaaaaaaccgtttatgttctgaccgcactgcaggattacattaataccaatctggttccgaccatcgataaaatcagctgtaaacagaccgaactgagcctggatctggccctgagcaaatatctgagcgatctgctgtttgtttttggtccgaatctgcaggatccggttagcaatagcatgaccattcaggcaattagccaggcatttggtggtaattatgaaaccctgctgcgtaccctgggttatgcaaccgaagattttgatgatctgctggaaagcgatagcattaccggtcagattatctatgttgatctgagcagctactatattatcgtgcgtgtgtattttccgatcctgaccgaaattcagcaggcatatatccaagaactgctgccggttagctttaataacgataatagcgaatggattagcatcgtgccgaactttattctggttcgtaataccctgattagcaacatcgaaattggcttttgcctgattaccaaacgtagcgtgatttgcaatcaggattatgcgaccccgatgaccaataatatgcgtgaatgtctgacaggtagcaccgaaaaatgtccgcgtgaactggttgttagcagccatgttccgcgttttgcactgagcaatggtgttctgtttgcaaattgtattagcgttacctgccagtgtcagaccaccggtcgtgccattagccagagcggtgaacagaccctgctgatgattgataataccacctgtccgaccgcagttctgggtaatgtgattattagcctgggtaaatatctgggcagcgtgaattataacagcgaaggtattgccattggtccgcctgtttttaccgataaagttgatattagctcccagatcagcagcatgaatcagagcctgcagcagagcaaagattatatcaaagaagcacagcgtctgctggataccgttaatccgagcctgattagtatgctgagcatgattattctgtacgttctgagcattgcaagcctgtgtattggtctgattacctttatcagcttcatcatcgtggaaaaaaaacgtgcagcatatagccgtctggaagatcgtcgtgttcgtccgaccagcagcggtgatctgtattatatcggcaccgaacagaaactgatcagcgaagaagatctgtaa

SEQ ID NO: 9 - DNA, 1620 bp, synthetic construct
  source 1..1620: mol_type = other DNA
atggactataaagacgatgacgacaaaatcctgcactatgaaaaactgagcaaaattggtctggttaaaggtgtgacccgcaaatacaaaatcaaaagcaatccgctgaccaaggacatcgtgatcaaaatgattccgaatgtgagcaatatgagccagtgtaccggtagcgttatggaaaactataaaacccgtctgaatggtattctgaccccgattaaaggtgcactggaaatctataagaacaacacccatgatctggttggtgatgttcgtctggcaggcgttattatggctggtgttgcaattggtattgcaaccgcagcacagattaccgcaggcgttgcactgtatgaagcaatgaaaaatgccgacaacatcaacaaactgaaaagcagcattgaaagcaccaatgaagcagttgttaaactgcaagaaaccgcagaaaaaaccgtttatgttctgaccgcactgcaggattacattaataccaatctggttccgaccatcgataaaatcagctgtaaacagaccgaactgagcctggatctggccctgagcaaatatctgagcgatctgctgtttgtttttggtccgaatctgcaggatccggttagcaatagcatgaccattcaggcaattagccaggcatttggtggtaattatgaaaccctgctgcgtaccctgggttatgcaaccgaagattttgatgatctgctggaaagcgatagcattaccggtcagattatctatgttgatctgagcagctactatattatcgtgcgtgtgtattttccgatcctgaccgaaattcagcaggcatatatccaagaactgctgccggttagctttaataacgataatagcgaatggattagcatcgtgccgaactttattctggttcgtaataccctgattagcaacatcgaaattggcttttgcctgattaccaaacgtagcgtgatttgcaatcaggattatgcgaccccgatgaccaataatatgcgtgaatgtctgacaggtagcaccgaaaaatgtccgcgtgaactggttgttagcagccatgttccgcgttttgcactgagcaatggtgttctgtttgcaaattgtattagcgttacctgccagtgtcagaccaccggtcgtgccattagccagagcggtgaacagaccctgctgatgattgataataccacctgtccgaccgcagttctgggtaatgtgattattagcctgggtaaatatctgggcagcgtgaattataacagcgaaggtattgccattggtccgcctgtttttaccgataaagttgatattagctcccagatcagcagcatgaatcagagcctgcagcagagcaaagattatatcaaagaagcacagcgtctgctggataccgttaatccgagcctgattagtatgctgagcatgattattctgtacgttctgagcattgcaagcctgtgtattggtctgattacctttatcagcttcatcatcgtggaaaaaaaacgtgcagcatatagccgtctggaagatcgtcgtgttcgtccgaccagcagcggtgatctgtattatatcggcaccgaacagaaactgatcagcgaagaagatctgtaa

SEQ ID NO: 10 - DNA, 1863 bp, synthetic construct
  source 1..1863: mol_type = other DNA
atggactataaagacgatgacgacaaaccggccgagaacaagaaggtccgctttgaaaatacgactagtgataagggtaagatcccttcgaaagtaatcaagagctactacggtaccatggatataaagaagataaacgagggactgttagactcaaagatactgtctgccttcaacacagtgatcgcgttactggggtctattgtcattatagtaatgaacataatgataatacaaaattacacccgctcgacagataaccaggcagtaatcaaggacgcgttacaaggtattcaacaacaaatcaaaggtcttgccgacaagattggcaccgaaatcggtccaaaggtcagcctcattgacacatcgagtaccataaccatcccagctaacatcggccttctgggctctaaaatttcccagagtacagcctctatcaacgagaacgtaaacgagaaatgcaagtttactttgcccccgctgaaaattcacgaatgcaacattagttgtccgaaccctttgccatttcgagagtaccgcccccaaacagagggcgtgtcaaacttggtaggactccccaataatatatgtctccagaagacttcaaatcaaatactgaagcctaagttgatatcatacaccctgccggtcgtcggccaaagcggcacatgtataacagacccgctccttgctatggacgagggttattttgcctacagccacctggagcgcatcgggtcatgtagccgtggcgtctctaagcagcgtattattggcgttggggaagtcttagaccgtggagacgaggttcctagtttattcatgaccaatgtgtggacacccccaaaccccaatacggtctatcactgttctgcagtatacaacaacgaattttactacgtgctttgtgcagtatcaaccgtaggcgatccgattctcaactccacgtattggagtggaagcttgatgatgacgagacttgctgttaagccaaagtcgaatggcggtggctacaatcaacatcaattagcgctccggagtattgagaagggtcgctacgacaaagttatgccctacggccctagtggtataaagcaaggggacacactgtacttccctgcagttggttttctggtgcgtaccgaattcaaatacaacgactcgaactgcccgataactaaatgccagtactccaagcctgaaaattgccgcctctcgatgggcatacgccctaattctcactacattttgagatcagggctgcttaagtacaatctgagtgacggcgaaaaccccaaggtggtgtttatcgagatatctgaccaaagactctccatcggtagcccttccaaaatctatgactcacttggtcagcccgtcttctatcaagcttcattcagttgggacacaatgattaagttcggagacgttctgacggtaaaccctctggtcgtgaactggcgcaataacaccgttataagccgtccaggacaatcgcagtgtcctcgtttcaacacctgcccagagatctgctgggaaggcgtctacaacgacgcgtttctgatcgatcggattaactggataagtgcaggggttttcttggacagcaatcaaaccgccgaaaaccctgttttcacagtgttcaaggacaacgaaatattgtaccgcgcccaacttgcttctgaggacacgaacgcacagaaaaccatcacaaattgctttctgttgaagaataagatttggtgtataagccttgtggaaatctacgacactggtgacaacgtcataagaccgaagttatttgcagtgaagatcccggagcaatgcacagaacagaaactgatcagcgaagaagatctgtaa

SEQ ID NO: 11 - DNA, 2109 bp, synthetic construct
  source 1..2109: mol_type = other DNA
atggactataaagacgatgacgacaaaatcctgcactatgaaaaactgagcaaaattggtctggttaaaggtgtgacccgcaaatacaaaatcaaaagcaatccgctgaccaaggacatcgtgatcaaaatgattccgaatgtgagcaatatgagccagtgtaccggtagcgttatggaaaactataaaacccgtctgaatggtattctgaccccgattaaaggtgcactggaaatctataagaacaacacccatgatctggttggtgatgttcgtctggcaggcgttattatggctggtgttgcaattggtattgcaaccgcagcacagattaccgcaggcgttgcactgtatgaagcaatgaaaaatgccgacaacatcaacaaactgaaaagcagcattgaaagcaccaatgaagcagttgttaaactgcaagaaaccgcagaaaaaaccgtttatgttctgaccgcactgcaggattacattaataccaatctggttccgaccatcgataaaatcagctgtaaacagaccgaactgagcctggatctggccctgagcaaatatctgagcgatctgctgtttgtttttggtccgaatctgcaggatccggttagcaatagcatgaccattcaggcaattagccaggcatttggtggtaattatgaaaccctgctgcgtaccctgggttatgcaaccgaagattttgatgatctgctggaaagcgatagcattaccggtcagattatctatgttgatctgagcagctactatattatcgtgcgtgtgtattttccgatcctgaccgaaattcagcaggcatatatccaagaactgctgccggttagctttaataacgataatagcgaatggattagcatcgtgccgaactttattctggttcgtaataccctgattagcaacatcgaaattggcttttgcctgattaccaaacgtagcgtgatttgcaatcaggattatgcgaccccgatgaccaataatatgcgtgaatgtctgacaggtagcaccgaaaaatgtccgcgtgaactggttgttagcagccatgttccgcgttttgcactgagcaatggtgttctgtttgcaaattgtattagcgttacctgccagtgtcagaccaccggtcgtgccattagccagagcggtgaacagaccctgctgatgattgataataccacctgtccgaccgcagttctgggtaatgtgattattagcctgggtaaatatctgggcagcgtgaattataacagcgaaggtattgccattggtccgcctgtttttaccgataaagttgatattagctcccagatcagcagcatgaatcagagcctgcagcagagcaaagattatatcaaagaagcacagcgtctgctggataccgttaatccgagcctgattagtatgctgagcatgattattctgggtagctcaggagcaagtccggcagcaccggcaccggcatcaccagcagctccagcacctagtgcaccggcaggcggtatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggacacatgggtagcgaacagaaactgatcagcgaagaagatctgtaa

SEQ ID NO: 12 - DNA, 2454 bp, synthetic construct
  source 1..2454: mol_type = other DNA
atggactataaagacgatgacgacaaaatcctgcactatgaaaaactgagcaaaattggtctggttaaaggtgtgacccgcaaatacaaaatcaaaagcaatccgctgaccaaggacatcgtgatcaaaatgattccgaatgtgagcaatatgagccagtgtaccggtagcgttatggaaaactataaaacccgtctgaatggtattctgaccccgattaaaggtgcactggaaatctataagaacaacacccatgatctggttggtgatgttcgtctggcaggcgttattatggctggtgttgcaattggtattgcaaccgcagcacagattaccgcaggcgttgcactgtatgaagcaatgaaaaatgccgacaacatcaacaaactgaaaagcagcattgaaagcaccaatgaagcagttgttaaactgcaagaaaccgcagaaaaaaccgtttatgttctgaccgcactgcaggattacattaataccaatctggttccgaccatcgataaaatcagctgtaaacagaccgaactgagcctggatctggccctgagcaaatatctgagcgatctgctgtttgtttttggtccgaatctgcaggatccggttagcaatagcatgaccattcaggcaattagccaggcatttggtggtaattatgaaaccctgctgcgtaccctgggttatgcaaccgaagattttgatgatctgctggaaagcgatagcattaccggtcagattatctatgttgatctgagcagctactatattatcgtgcgtgtgtattttccgatcctgaccgaaattcagcaggcatatatccaagaactgctgccggttagctttaataacgataatagcgaatggattagcatcgtgccgaactttattctggttcgtaataccctgattagcaacatcgaaattggcttttgcctgattaccaaacgtagcgtgatttgcaatcaggattatgcgaccccgatgaccaataatatgcgtgaatgtctgacaggtagcaccgaaaaatgtccgcgtgaactggttgttagcagccatgttccgcgttttgcactgagcaatggtgttctgtttgcaaattgtattagcgttacctgccagtgtcagaccaccggtcgtgccattagccagagcggtgaacagaccctgctgatgattgataataccacctgtccgaccgcagttctgggtaatgtgattattagcctgggtaaatatctgggcagcgtgaattataacagcgaaggtattgccattggtccgcctgtttttaccgataaagttgatattagctcccagatcagcagcatgaatcagagcctgcagcagagcaaagattatatcaaagaagcacagcgtctgctggataccgttaatccgagcctgattagtatgctgagcatgattattctgggtagctcaggagcaagtccggcagcaccggcaccggcatcaccagcagctccagcacctagtgcaccggcaggcggtatggcagaaattggcaccggttttccgtttgatccgcattatgttgaagttctgggtgaacgtatgcattatgtggatgttggtccgcgtgatggtacaccggttctgtttctgcatggtaatccgaccagcagctatgtttggcgtaacattattccgcatgttgcaccgacacatcgttgtattgcaccggatctgattggtatgggtaaaagcgataaacctgatctgggctatttctttgatgatcatgtgcgttttatggacgcctttattgaagcactgggtttagaagaagttgtgctggttattcatgattggggtagtgccctgggttttcattgggcaaaacgtaatccggaacgtgttaaaggtattgcctttatggaattcattcgtccgattccgacctgggatgaatggcctgaatttgcacgtgaaacctttcaggcatttcgtaccaccgatgtgggtcgtaaactgattattgatcagaacgtttttatcgaaggcaccctgccgatgggtgttgttcgtccgctgaccgaagttgaaatggatcattatcgtgaaccgtttctgaatccggttgatcgcgaaccgctgtggcgttttccgaatgaactgccgattgccggtgaacctgcaaatattgttgcactggttgaagagtatatggattggctgcatcagagtccggttccgaaactgctgttttggggcacaccgggtgttctgattccgcctgcagaagcagcacgtctggcaaaaagcctgccgaattgtaaagcagttgatattggtccgggtctgaatctgctgcaagaagataatccagatctgatcggtagcgaaattgcacgttggctgagcaccctggaaattagcggtcacatgggtagcgaacagaaactgatcagcgaagaagatctgtaa

SEQ ID NO: 13 - DNA, 2292 bp, synthetic construct
  source 1..2292: mol_type = other DNA
atggactataaagacgatgacgacaaacacatgggtagcatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggaggtagctcaggagcaagtccggcagcaccggcaccggcatcaccagcagctccagcacctagtgcaccggcaggcggtcaaaattacacccgctcgacagataaccaggcagtaatcaaggacgcgttacaaggtattcaacaacaaatcaaaggtcttgccgacaagattggcaccgaaatcggtccaaaggtcagcctcattgacacatcgagtaccataaccatcccagctaacatcggccttctgggctctaaaatttcccagagtacagcctctatcaacgagaacgtaaacgagaaatgcaagtttactttgcccccgctgaaaattcacgaatgcaacattagttgtccgaaccctttgccatttcgagagtaccgcccccaaacagagggcgtgtcaaacttggtaggactccccaataatatatgtctccagaagacttcaaatcaaatactgaagcctaagttgatatcatacaccctgccggtcgtcggccaaagcggcacatgtataacagacccgctccttgctatggacgagggttattttgcctacagccacctggagcgcatcgggtcatgtagccgtggcgtctctaagcagcgtattattggcgttggggaagtcttagaccgtggagacgaggttcctagtttattcatgaccaatgtgtggacacccccaaaccccaatacggtctatcactgttctgcagtatacaacaacgaattttactacgtgctttgtgcagtatcaaccgtaggcgatccgattctcaactccacgtattggagtggaagcttgatgatgacgagacttgctgttaagccaaagtcgaatggcggtggctacaatcaacatcaattagcgctccggagtattgagaagggtcgctacgacaaagttatgccctacggccctagtggtataaagcaaggggacacactgtacttccctgcagttggttttctggtgcgtaccgaattcaaatacaacgactcgaactgcccgataactaaatgccagtactccaagcctgaaaattgccgcctctcgatgggcatacgccctaattctcactacattttgagatcagggctgcttaagtacaatctgagtgacggcgaaaaccccaaggtggtgtttatcgagatatctgaccaaagactctccatcggtagcccttccaaaatctatgactcacttggtcagcccgtcttctatcaagcttcattcagttgggacacaatgattaagttcggagacgttctgacggtaaaccctctggtcgtgaactggcgcaataacaccgttataagccgtccaggacaatcgcagtgtcctcgtttcaacacctgcccagagatctgctgggaaggcgtctacaacgacgcgtttctgatcgatcggattaactggataagtgcaggggttttcttggacagcaatcaaaccgccgaaaaccctgttttcacagtgttcaaggacaacgaaatattgtaccgcgcccaacttgcttctgaggacacgaacgcacagaaaaccatcacaaattgctttctgttgaagaataagatttggtgtataagccttgtggaaatctacgacactggtgacaacgtcataagaccgaagttatttgcagtgaagatcccggagcaatgcacagaacagaaactgatcagcgaagaagatctgtaa

SEQ ID NO: 14 - DNA, 2637 bp, synthetic construct
  source 1..2637: mol_type = other DNA
atggactataaagacgatgacgacaaacacatgggtagcatggcagaaattggcaccggttttccgtttgatccgcattatgttgaagttctgggtgaacgtatgcattatgtggatgttggtccgcgtgatggtacaccggttctgtttctgcatggtaatccgaccagcagctatgtttggcgtaacattattccgcatgttgcaccgacacatcgttgtattgcaccggatctgattggtatgggtaaaagcgataaacctgatctgggctatttctttgatgatcatgtgcgttttatggacgcctttattgaagcactgggtttagaagaagttgtgctggttattcatgattggggtagtgccctgggttttcattgggcaaaacgtaatccggaacgtgttaaaggtattgcctttatggaattcattcgtccgattccgacctgggatgaatggcctgaatttgcacgtgaaacctttcaggcatttcgtaccaccgatgtgggtcgtaaactgattattgatcagaacgtttttatcgaaggcaccctgccgatgggtgttgttcgtccgctgaccgaagttgaaatggatcattatcgtgaaccgtttctgaatccggttgatcgcgaaccgctgtggcgttttccgaatgaactgccgattgccggtgaacctgcaaatattgttgcactggttgaagagtatatggattggctgcatcagagtccggttccgaaactgctgttttggggcacaccgggtgttctgattccgcctgcagaagcagcacgtctggcaaaaagcctgccgaattgtaaagcagttgatattggtccgggtctgaatctgctgcaagaagataatccagatctgatcggtagcgaaattgcacgttggctgagcaccctggaaattagcggtggtagctcaggagcaagtccggcagcaccggcaccggcatcaccagcagctccagcacctagtgcaccggcaggcggtcaaaattacacccgctcgacagataaccaggcagtaatcaaggacgcgttacaaggtattcaacaacaaatcaaaggtcttgccgacaagattggcaccgaaatcggtccaaaggtcagcctcattgacacatcgagtaccataaccatcccagctaacatcggccttctgggctctaaaatttcccagagtacagcctctatcaacgagaacgtaaacgagaaatgcaagtttactttgcccccgctgaaaattcacgaatgcaacattagttgtccgaaccctttgccatttcgagagtaccgcccccaaacagagggcgtgtcaaacttggtaggactccccaataatatatgtctccagaagacttcaaatcaaatactgaagcctaagttgatatcatacaccctgccggtcgtcggccaaagcggcacatgtataacagacccgctccttgctatggacgagggttattttgcctacagccacctggagcgcatcgggtcatgtagccgtggcgtctctaagcagcgtattattggcgttggggaagtcttagaccgtggagacgaggttcctagtttattcatgaccaatgtgtggacacccccaaaccccaatacggtctatcactgttctgcagtatacaacaacgaattttactacgtgctttgtgcagtatcaaccgtaggcgatccgattctcaactccacgtattggagtggaagcttgatgatgacgagacttgctgttaagccaaagtcgaatggcggtggctacaatcaacatcaattagcgctccggagtattgagaagggtcgctacgacaaagttatgccctacggccctagtggtataaagcaaggggacacactgtacttccctgcagttggttttctggtgcgtaccgaattcaaatacaacgactcgaactgcccgataactaaatgccagtactccaagcctgaaaattgccgcctctcgatgggcatacgccctaattctcactacattttgagatcagggctgcttaagtacaatctgagtgacggcgaaaaccccaaggtggtgtttatcgagatatctgaccaaagactctccatcggtagcccttccaaaatctatgactcacttggtcagcccgtcttctatcaagcttcattcagttgggacacaatgattaagttcggagacgttctgacggtaaaccctctggtcgtgaactggcgcaataacaccgttataagccgtccaggacaatcgcagtgtcctcgtttcaacacctgcccagagatctgctgggaaggcgtctacaacgacgcgtttctgatcgatcggattaactggataagtgcaggggttttcttggacagcaatcaaaccgccgaaaaccctgttttcacagtgttcaaggacaacgaaatattgtaccgcgcccaacttgcttctgaggacacgaacgcacagaaaaccatcacaaattgctttctgttgaagaataagatttggtgtataagccttgtggaaatctacgacactggtgacaacgtcataagaccgaagttatttgcagtgaagatcccggagcaatgcacagaacagaaactgatcagcgaagaagatctgtaa

(19) United States
(12) Patent Application Publication
(10) Pub. No.: US 2026/0130982 A1
(43) Pub. Date: May 14, 2026

(54) METHODS FOR ASSEMBLING PROTEIN-CONJUGATED NANOCARRIER VACCINES

(71) Applicants: Northwestern University, Evanston, IL (US); Cornell University, Ithaca, NY (US)

(72) Inventors: Neha Prashant Kamat, Evanston, IL (US); Vivian Hu, Evanston, IL (US); Susan Daniel, Ithaca, NY (US); Hector Aguilar-Carreno, Ithaca, NY (US); Ekaterina Selivanovitch, Ithaca, NY (US); Shahrzad Ezzatpour, Ithaca, NY (US)

(21) Appl. No.: 19/120,101

(22) PCT Filed: Oct. 13, 2023
(86) PCT No.: PCT/US2023/076919

§ 371 Date: Apr. 10, 2025

Related U.S. Application Data
(60) Provisional application No. 63/379,418, filed on Oct. 13, 2022.

(51) Int. Cl. A61K 39/155; A61K 9/127; A61K 9/51; A61K 39/00; A61K 39/385; A61K 47/54; A61K 47/62; A61K 47/69; A61P 31/14; C07K 14/005; C12N 7/00

(52) U.S. Cl. CPC A61K 39/155; A61K 9/127; A61K 9/5123; A61K 39/385; A61K 47/543; A61K 47/62; A61K 47/6911; A61P 31/14; C07K 14/005; C12N 7/00; A61K 2039/5258; A61K 2039/55555; A61K 2039/55572; A61K 2039/575; A61K 2039/6018; C07K 2319/03; C07K 2319/90; C12N 2760/18222; C12N 2760/18234

(57) ABSTRACT

Provided herein are vaccine compositions for preparing nanocarriers comprising NiVF and NiVG virus proteins. Methods for preparing and using the nanocarriers for eliciting neutralizing antibodies or treating a Nipah virus infection are also described herein.

### CROSS-REFERENCE TO RELATED APPLICATIONS

This application claims priority to U.S. Provisional Application No. 63/379,418 filed on Oct. 13, 2022, the content of which is incorporated by reference in its entirety.

### STATEMENT REGARDING FEDERALLY SPONSORED RESEARCH

This invention was made with government support under grant number FA9550-19-1-0039 awarded by the Department of Energy. The government has certain rights in the invention.

### REFERENCE TO AN ELECTRONIC SEQUENCE LISTING

The contents of the electronic sequence listing (702581.02433.xml; Size: 32,522 bytes; and Date of Creation: Oct. 13, 2023) is herein incorporated by reference in its entirety.

### BACKGROUND OF THE INVENTION

In 2018, the World Health Organization called for accelerated R&D on a list of priority diseases, including the Nipah virus infection, with no efficacious drugs or vaccines that have potential to cause future public health crises. Vaccines remain an effective protective tool against future threats, but many current vaccines have limitations restricting global impact. Current vaccination strategies have limitations in speed, safety, potency, and storage: cell-derived vaccines take days to months to make; attenuated or inactivated viruses pose a risk of conversion to the pathogenic virus; recombinant proteins are weakly immunogenic; and gene-based vaccines that include mRNA are highly unstable and require cold chain storage.

Cell-free protein synthesis systems (CFPS), which use extracted protein expression machinery from cells to synthesize proteins in vitro, are a promising tool for vaccine development because they can rapidly produce clinically-relevant amounts of protein, can be freeze-dried for distribution, and would prevent the need for cold chain storage. Yet, most viral vaccine targets are membrane proteins, which can be difficult to produce with CFPS alone. We and others have shown that adding liposomes, or nanoparticles containing a lipid bilayer, to these systems provides a scaffold to promote the co-translational expression and folding of membrane proteins, resulting in one-step assembly of lipid nanoparticles with membrane-integrated proteins. This technology has the potential to assemble potent viral mimetic vaccines that present viral antigens and adjuvants for emerging outbreaks. Compared to many current vaccines, this platform bypasses the need for cold chain biomanufacturing and storage and can be quickly assembled once a potential threat is identified.

Thus, there is potential for assembling lipid nanocarrier with membrane proteins to prepare Nipah virus vaccines.

### SUMMARY OF THE INVENTION

In a first aspect, provided herein is a vaccine composition comprising a nanocarrier comprising a membrane; a cell-free protein synthesis system; and at least one of a nucleic acid template encoding a NiVG protein comprising a transmembrane domain and a nucleic acid template encoding a NiVF protein comprising a transmembrane domain; wherein the NiVG transmembrane domain and the NiVF transmembrane domain are able to integrate into the nanocarrier membrane. In embodiments, the composition comprises the nucleic acid template encoding the NiVG protein and the nucleic acid template encoding the NiVF protein. In embodiments, the NiVF protein comprises SEQ ID NO: 1 or a sequence having at least 90% identity thereto, and wherein the NiVG protein comprises SEQ ID NO: 3 or a sequence having at least 90% identity thereto. In embodiments, the NiVF protein comprises a deletion of the signal peptide sequence. In embodiments, the NiVF protein comprises SEQ ID NO: 2 or a sequence having at least 90% identity thereto.

In embodiments, the nanocarrier is a liposome, a polymersome, or a lipid nanoparticle. In embodiments, the nanocarrier is a liposome. In embodiments, the liposome membrane comprises phosphatidylcholine (PC) headgroups. In embodiments, the liposome membrane comprises a mixture of PC headgroups and phosphoethanolamine (PE) headgroups; optionally wherein the PC and PE headgroups are comprised at a ratio of about 4:1 PC:PE. In embodiments, the liposome membrane comprises a mixture of PC headgroups, PE headgroups, and phosphatidylserine (PS) headgroups; optionally wherein the PC, PE, and PS headgroups are comprised at a ratio of about 15:4:1 PC:PE:PS. In embodiments, the liposome membrane further comprises a lipid adjuvant; optionally wherein the lipid adjuvant is monophosphoryl lipid A (MPLA) at a concentration of between about 0.004% and about 0.4%.

In embodiments, the nanocarrier further comprises a cargo molecule. In embodiments, the cargo molecule comprises a polynucleotide, a polypeptide, an active pharmaceutical ingredient, or a therapeutic agent.

In another aspect, provided herein is a vaccine nanoparticle prepared from the composition disclosed herein, wherein the vaccine nanoparticle is prepared by expressing the nucleic acid template encoding the NiVG protein or the nucleic acid template encoding the NiVF protein; optionally wherein the method comprises co-expressing the nucleic acid template encoding the NiVG protein and the nucleic acid template encoding the NiVF protein.

In another aspect, provided herein is a vaccine nanoparticle comprising the nanocarrier and at least one of the NiVG protein or the NiVF fusion protein disclosed herein, wherein at least one of the NiVG transmembrane domain and the NiVF transmembrane domain is integrated into the nanocarrier membrane. In embodiments, the vaccine nanoparticle comprises both the NiVG protein and the NiVF protein and wherein the NivG transmembrane domain and the NiVF transmembrane domain are integrated into the nanocarrier membrane.

In another aspect, provided herein is a vaccine composition comprising a nanocarrier comprising a membrane; a cell-free protein synthesis system; and at least one of a nucleic acid template encoding a NiVG fusion protein and a nucleic acid template encoding a NiVF fusion protein, wherein the membrane comprises at least one of a first modified lipid and a second modified lipid; and wherein the NiVG fusion protein comprises a first protein tag that conjugates to the first modified lipid and a NiVG Nipah virus protein, and wherein the NiVF fusion protein comprises a second protein tag that conjugates to the second modified lipid and a NiVF Nipah virus protein.

In embodiments, the composition comprises the nucleic acid template encoding the NiVG fusion protein and the nucleic acid template encoding the NiVF fusion protein. In embodiments, the NiVF Nipah virus protein comprises SEQ ID NO: 1 or a sequence having at least 90% identity thereto, and wherein the NiVG Nipah virus protein comprises SEQ ID NO: 3 or a sequence having at least 90% identity thereto. In embodiments, the NiVF protein comprises a deletion of the signal peptide sequence. In embodiments, the NiVF protein comprises SEQ ID NO: 2 or a sequence having at least 90% identity thereto. In embodiments, the first modified lipid and the second modified lipid are the same. In embodiments, the first modified lipid and the second modified lipid are different. In embodiments, the first protein tag and the second protein tag are self-labeling enzymes. In embodiments, at least one of the first and second modified lipid comprises benzylguanine and at least one of the self-labeling enzyme is a SNAP-tag. In embodiments, at least one of the first and second modified lipid comprises a HaloTag ligand and at least one of the first and second self-labeling enzyme is a HaloTag.

In embodiments, the nanocarrier is a liposome, a polymersome, or a lipid nanoparticle. In embodiments, the nanocarrier is a liposome. In embodiments, the liposome membrane comprises phosphatidylcholine (PC) headgroups. In embodiments, the liposome membrane comprises a mixture of PC headgroups and phosphoethanolamine (PE) headgroups; optionally wherein the PC and PE headgroups are comprised at a ratio of about 4:1 PC:PE. In embodiments, the liposome membrane comprises a mixture of PC headgroups, PE headgroups, and phosphatidylserine (PS) headgroups; optionally wherein the PC, PE, and PS headgroups are comprised at a ratio of about 15:4:1 PC:PE:PS. In embodiments, the liposome membrane further comprises a lipid adjuvant; optionally wherein the lipid adjuvant is monophosphoryl lipid A (MPLA) at a concentration of between about 0.004% and about 0.4%.

In embodiments, the nanocarrier further comprises a cargo molecule. In embodiments, the cargo molecule comprises a polynucleotide, a polypeptide, an active pharmaceutical ingredient, or a therapeutic agent.

In another aspect, provided herein is a vaccine nanoparticle prepared from the composition disclosed herein, wherein the vaccine nanoparticle is prepared by expressing the nucleic acid template encoding the NiVG fusion protein or the nucleic acid template encoding the NiVF fusion protein; optionally wherein the method comprises co-expressing the nucleic acid template encoding the NiVG fusion protein and the nucleic acid template encoding the NiVF fusion protein.

In another aspect, provided herein is a vaccine nanoparticle comprising the nanocarrier and at least one of the NiVG fusion protein and the NiVF fusion protein disclosed herein, wherein the at least one protein tag is associated with the modified lipid. In embodiments, the vaccine nanoparticle comprises both the NiVG fusion protein and the NiVF fusion protein and wherein the first and second protein tags are associated with the first and second modified lipid.

In another aspect, provided herein is a method for eliciting neutralizing antibodies in a subject, the method comprising administering the vaccine nanoparticle disclosed herein to the subject.

In another aspect, provided herein is a method for treating a Nipah virus infection in a subject, the method comprising administering to the subject a therapeutically effective amount of the vaccine nanoparticle disclosed herein.

In another aspect, provided herein is a method for preparing a vaccine nanoparticle, the method comprising expressing at least one of the nucleic acid template encoding the NiVG protein and the nucleic acid template encoding the NiVF protein in the composition disclosed herein. In embodiments, the method comprises co-expressing the nucleic acid template encoding the NiVG protein and the nucleic acid template encoding the NiVF protein.

In another aspect, provided herein is a method for preparing a vaccine nanoparticle, the method comprising expressing at least one of the nucleic acid template encoding the NiVG fusion protein and the nucleic acid template encoding the NiVF fusion protein in the composition disclosed herein. In embodiments, the method comprises co-expressing the nucleic acid template encoding the NiVG fusion protein and the nucleic acid template encoding the NiVF fusion protein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1E. Viral membrane proteins can be cotranslationally integrated into vesicles using cell-free protein synthesis. (A,B) Schematic of experimental design: (A) Presentation of viral membrane proteins on lipid vesicles enables assembly of viral mimetics. (B) Using cell-free protein synthesis systems, membrane proteins can be rapidly synthesized in reactions. Supplementing reactions with hydrophobic supplements such as liposomes leads to cotranslational integration of the synthesized proteins. (C) Design of genes for two single-pass transmembrane proteins: Nipah virus fusion (NiV F) and Nipah virus attachment (NiV G). (D) Western blot of purified DOPC vesicles after cell-free expression reactions reveals full-length proteins NiV F, NiV F ΔSP, and NiV F proteins are present and membrane associated. Antibodies against a C-terminal Myc tag were used for blot analysis. (E) Deletion of the signal peptide sequence in Type I transmembrane protein NiV F (NiV F ΔSP) enhances NiV F expression by roughly 11-fold. Error bars represent the standard deviation for n=3 independent replicates.

FIGS. 2A-2E. Alteration of liposome membrane composition enhances NiV F and NiV G association with liposome membranes. (A) Variation of phospholipid headgroups (phosphatidylcholine (PC), phosphoethanolamine (PE), and phosphatidylserine (PS)) were investigated for effects on vaccine assemblyprotein association with liposomes. (B) NiV F ΔSP was expressed in PC, 4:1 PC:PE (PC/PE), and 15:4:1 PC:PE:PS (PC/PE/PS) liposomes. PC/PE and PC/PE/PS compositions led to 6.3- and 8.9-fold improvements in NiV F ΔSP expression and integration. (C) NiV G expression in PC, PC/PE, and PC/PE/PS liposomes yielded increased NiV G integration of 1.8- and 1.5-fold in PC/PE and PC/PE/PS liposomes respectively. (D) Co-expression of NiV F ΔSP and NiV G in the presence of PC, PC/PE, and PC/PE/PS liposomes results in detection of both proteins associated with vesicles, assessed via western blot analysis. (E) Semi-quantitative western blot analysis reveals that the inclusion of MPLA lipid at amounts of 0%, 0.004%, 0.04%, and 0.4% in PC/PE/PS liposomes does not affect the expression of NiV F ΔSP and NiV G. Error bars represent the standard deviation for n=4, n=3, or n=2 independent replicates.

FIGS. 3A-3C. Cell-free expressed viral membrane proteins maintain native folding conformation. (A) Flow cytometry was used to detect native folding and orientation of NiV F ΔSP and NiV G in fluorescent (Cy5.5) PC/PE/PS liposomes. Protein-mediated binding of liposomes to beads were detected with monoclonal conformational antibodies for NiV F or NiV G. (B) NiV F ΔSP and NiV G expressed in fluorescent PC/PE/PS liposomes as well as an empty vesicle control were incubated with beads functionalized with a monoclonal antibody detecting NiV F conformation (Mab 92). PC/PE/PS liposomes containing NiV F ΔSP bind beads with the NiV F conformational antibody (Mab 92) with a ˜36-fold increase in median fluorescence intensity relative to the empty vesicle control compared to ˜1.6-fold for liposomes containing NiV G. The NiV F ΔSP sample demonstrated a ˜36-fold increase in median fluorescence intensity, normalized to the vesicle control and is significantly more than the NiV G sample which had only a 1.6-fold increase. The NiV G curve completely overlaps the Vesicle Control curve. (C) PC/PE/PS liposomes containing NiV G bind beads with the NiV G conformational antibody (Mab 213) with a 65-fold increase in median fluorescence intensity relative to the empty vesicle control compared to a 6.7-fold increase for NiV F ΔSP-containing vesicles. Error bars represent the standard deviation for n=3 independent replicates.

FIG. 4 illustrates conjugation of cell-free expressed viral membrane proteins to liposomes using self-labeling protein tags.

FIG. 5 outlines the experimental design of a mouse study for the lipid vesicle-based NiV vaccine.

FIG. 6 shows lipid vesicle-based NiV vaccine elicits neutralizing antibodies in mice.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

Nipah virus is a bat-borne, zoonotic virus of the genus Henipavirus that causes Nipah virus infection in humans and other animals, a disease with a very high mortality rate (40-75%). Numerous disease outbreaks caused by Nipah virus have occurred in North East Africa and Southeast Asia. Like other henipaviruses, the Nipah virus genome is a single (non-segmented) negative-sense, single-stranded RNA of over 18 kb. The enveloped virus particles are variable in shape, and can be filamentous or spherical; they contain a helical nucleocapsid. Six structural proteins are generated: N (nucleocapsid), P (phosphoprotein), M (matrix), F (fusion), G (glycoprotein) and L (RNA polymerase). The P open reading frame also encodes three nonstructural proteins, C, V and W.

There are two envelope glycoproteins. The G glycoprotein (NiV G) ectodomain assembles as a homotetramer to form the viral anti-receptor or attachment protein, which binds to the receptor on the host cell. The G protein head domain is highly antigenic, inducing head-specific antibodies in primate models. As such, it is a prime target for vaccine development as well as antibody therapy. The F glycoprotein (NiV F) forms a trimer, which mediates membrane fusion.

Cell-free protein synthesis systems can rapidly create protein-conjugated membrane-based nanocarriers. Using this approach, multiple types of functional binding proteins, including affibodies, computationally designed proteins, as well as scFvs, can be expressed cell-free and conjugated to liposomes in one-pot. This technique can be further expanded to other nanocarriers, including polymersomes and lipid nanoparticles, and is amenable to multiple conjugation strategies, including surface attachment to and integration into nanocarrier membranes. These methods are further leveraged in vitro demonstrating rapidly designed bispecific artificial antigen presenting cells and enhanced delivery of lipid nanocarrier cargo. This workflow enables the rapid generation of membrane-based delivery systems and bolster the ability to create cell-mimetic therapeutics.

Cell-free expressed proteins may be conjugated to lipid-based nanocarriers. Conjugation of proteins may be through chemical conjugation, for example chemical or self-labeling conjugation or via hydrophobic transmembrane domain insertion. Different conjugation methods may allow for a protein of interest to be conjugated to a lipid-based nanocarrier in a site-specific manner. Chemical conjugation may allow for a protein to be conjugated to the surface of lipid-based nanocarriers. Conjugation with a transmembrane domain allows for a protein to be inserted into the membrane of lipid-based nanocarriers.

In a first aspect, provided herein is a vaccine composition comprising a nanocarrier comprising a membrane; a cell-free protein synthesis system; and at least one of a nucleic acid template encoding a NiVG protein comprising a transmembrane domain and a nucleic acid template encoding a NiVF protein comprising a transmembrane domain; wherein the NiVG transmembrane domain and the NiVF transmembrane domain are able to integrate into the nanocarrier membrane. The composition may comprise both the nucleic acid template encoding the NiVG protein and the nucleic acid template encoding the NiVF protein. The NiVG protein and NiVF protein includes immunogenic fragments thereof.

The term “vaccine,” as used herein, refers to a composition that includes an antigen. Vaccine may also include a biological preparation that improves immunity to a particular disease. A vaccine may typically contain an agent, referred to as an antigen, that resembles a disease-causing microorganism, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the subcutaneous, oral, oronasal, or intranasal routes of administration. A vaccine of the present invention includes a suitable antigen to stimulate an immune response against Nipah virus in a subject or patient or to treat a Nipah virus infection.

The term “nanocarrier” refers to a nanomaterial used as a transport module for another substance. Nanocarriers include, without limitation, liposomes, micelles, nanoparticles, extracellular vesicles (e.g. vesicles derived from mammalian cells), outer membrane vesicles (e.g. vesicles derived from bacteria), polymersomes, and dendrimers. The compositions described herein may also include microparticles and other therapeutic materials, such as giant unilamellar vesicles, inorganic beads, hydrogels, and cell membranes.

Lipid-based nanocarriers are colloidal carriers for bioactive organic molecules. A “lipid layer,” “lipid structure,” or “lipid membrane” means a continuous, self-assembled barrier comprising a plurality of amphiphilic lipids. In some embodiments, the lipid layer comprises a single layer of amphiphilic lipids, e.g., a micelle or a reverse micelle, having a hydrophilic surface and a hydrophobic surface. In other embodiments, the lipid layer is a lipid bilayer comprising two layers of amphiphilic lipids having an inner-hydrophilic surface, an outer-hydrophilic surface, and a hydrophobic core disposed between the inner-hydrophilic surface and the outer-hydrophilic surface, e.g., a liposome, a lipid nanoparticle, a cell, a cellular organelle, or a 2-dimensional membrane.

“Amphiphilic lipid” means any chemical compound having both hydrophilic and hydrophobic properties and typically composed of a polar head group and lipophilic tail. The polar head group may charged or uncharged. Suitably, the polar head groups may comprise anionic head groups (such as carboxylates, sulfates, sulfonates, or phosphates), cationic head groups (such as ammoniums), or uncharged head groups (such as alcohols). The lipophilic tail is typically a saturated or unsaturated alkyl or a saturated or unsaturated alkylene having at least four carbon atoms, suitably between 6 and 24 carbon atoms. Exemplary amphiphilic lipids include, without limitation, phospholipids (e.g., sphingomyelins or phosphoglycerides such as phosphatidylserines, phosphatidylethanolamines, phosphatidylinositols, or phosphatidylcholines), glycolipids, fatty acids, amphiphilic di-block copolymers, amphiphilic tri-block copolymers, amphiphilic dendrimers, amphiphilic dendrons, or peptide amphiphiles.

The nanocarriers used herein may be liposomes having homogenous or heterogenous phospholipid head groups. The liposome phospholipids may comprise all phosphatidylcholine (PC) headgroups.

Alternatively, the liposome phospholipids may comprise a mixture of PC headgroups and phosphoethanolamine (PE) headgroups. The PC and PE headgroups may be comprised at a ratio of between about 3:1 and 5:1 PC:PE and any ratio or range in between, e.g. about 3:1, 3.5:1, 4:1, 4.5:1, 5:1, etc. In exemplary embodiments, the PC and PE headgroups are comprised at a ratio of about 4:1 PC:PE.

Alternatively, the liposome phospholipids may comprise a mixture of PC headgroups, PE headgroups, and phosphatidylserine (PS) headgroups. In exemplary embodiments, PC, PE, and PS headgroups may be comprised at a ratio of about 15:4:1 PC:PE:PS. However, other ratios may be used, e.g. about 16:4:1, 14:4:1, 15:5:1, 15:3:1, etc.

The liposome membrane may further comprise a lipid adjuvant. The term “adjuvant” refers to a compound or mixture that is present in an immunogenic composition or vaccine and enhances the immune response to an antigen present in the immunogenic composition or vaccine. For example, an adjuvant may enhance the immune response to the NiVG or NiVF protein as disclosed herein. The lipid adjuvant may comprise monophosphoryl lipid A (MPLA). The MPLA may be comprised in the membrane at a concentration of between about 0.004% and about 0.4%, and any concentrations and ranges in between, including about 0.04%.

Once assembled, the nanocarrier is incubated with a cell-free protein synthesis system including the components necessary for transcription and translation of a protein; and a nucleic acid template encoding a fusion protein and having the regulatory components required for protein transcription and translation, such as a promoter and ribosome binding site.

A “cell-free protein synthesis system,” “CFPS reaction mixture,” or “cell-free system” typically contains a crude or partially-purified cell extract and a suitable reaction buffer for promoting cell-free protein synthesis from a nucleic acid template. The nucleic acid template may include an RNA template and/or a DNA template. The DNA template may include an open reading frame operably linked to a promoter element for a DNA-dependent RNA polymerase. Additional NTP's and divalent cation cofactor may be included in the cell-free system. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

Cell-free systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties).

Cell-free systems may comprise a cellular extract from a host strain. Because cell-free protein synthesis systems exploit an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Patent Application Publication No. 2014/0295492 and U.S. Patent Application Publication No. 2016/0060301, the contents of which are incorporated by reference in their entireties. The cellular extract of the platform may be prepared from a cell culture of a prokaryote (e.g., E. coli). While E. coli is exemplified herein, the bacterial species is not intended to be limiting. Other bacterial species suitable for the compositions and methods disclosed herein include but are not limited to (e.g., Bacillis species such as Bacillus subtilis, Vibrio species such as Vibrio natrigens, Pseudomonas species, etc.). In some embodiments, the cell culture is in stationary phase. In some embodiments, stationary phase may be defined as the cell culture having an OD600 of greater than about 3.0, 3.5, 4.0, 4.5, 5.0, 5.5, 6.0, 6.5, 7.0, 7.5, 8.0, or having an OD600 within a range bounded by any of these values. Further methods for preparing a cell-free system are disclosed in International Patent Application Publication No. WO2020185451A2, the contents of which are incorporated by reference in its entirety.

The cell extract may be prepared by lysing the cells of the cell culture and isolating a fraction from the lysed cells. For example, the cell extract may be prepared by lysing the cells of the cell culture and subjecting the lysed cells to centrifugal force, and isolating a fraction after centrifugation.

The nucleic acid template may comprise an expression template, a translation template, or both an expression template and a translation template, including but not limited to plasmid DNA, linear DNA or mRNA. The template may comprise a construct configured to express the fusion protein. As used herein, the term “construct” refers to a recombinant polynucleotide, i.e., a polynucleotide that was formed artificially by combining at least two polynucleotide components from different sources (natural or synthetic). For example, the constructs described herein comprise a polynucleotide encoding the fusion protein disclosed herein, operably linked to a promoter that (1) is associated with another gene found within the same genome, (2) from the genome of a different species, or (3) is synthetic. Constructs can be generated using conventional recombinant DNA methods. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. The system may comprise one or more polymerases capable of generating a translation template from an expression template.

The terms “nucleic acid,” “nucleic acid sequence,” “polynucleotide,” and “polynucleotide sequence,” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated. The term “promoter” refers to a cis-acting DNA sequence that directs RNA polymerase and other trans-acting transcription factors to initiate RNA transcription from the DNA template that includes the cis-acting DNA sequence.

The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

The term “transcription factor” refers to a protein that regulates transcription of another protein, typically by interacting with one or more cis-acting DNA sequence in or near the promoter for the other protein. A transcription factor may increase expression or decrease expression depending upon whether the transcription factor is activated or deactivated. A transcription factor may become activated or deactivated by an interaction with another molecule (e.g., a target molecule as described above).

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, bacteriophage polymerases such as, but not limited to, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The vector may be a recombinant vector (e.g., a recombinant expression vector) comprising the nucleic acid sequence or construct encoding the fusion protein described herein. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” or “recombinant expression vectors.” One type of vector is a “plasmid”, which refers to a circular double stranded DNA loop into which additional DNA segments may be ligated, specifically exogenous DNA segments encoding the fusion protein.

Cell free reaction mixtures may be incubated with nanocarriers at a specified ratio for 2 hours at room temperature or overnight at 4° C. An advantage of the compositions and methods described herein is that the proteins of interest, e.g. NiVG and NiVF can be conjugated to a lipid-based nanocarrier in a short period of time, for example the total time of the method may be less than 10 hrs and not include any living cells.

The NiVF and NiVG proteins each comprise a transmembrane domain. A “transmembrane domain” refers to a membrane-spanning protein domain. In embodiments, the nanocarriers of the invention are suitable for integration of the NiVF and NiVG transmembrane domains into their membranes.

The NiVF protein may comprise SEQ ID NO: 1 or a sequence having at least 66%, 80%, 81%, 82%, 83%, 84%, 85%, 86%, 87%, 88%, 89%, 90%, 91%, 92%, 93%, 94%, 95%, 96%, 97%, 98% or 99% identity thereto. The NiVG protein may comprise SEQ ID NO: 3 or a sequence having at least 66%, 80%, 81%, 82%, 83%, 84%, 85%, 86%, 87%, 88%, 89%, 90%, 91%, 92%, 93%, 94%, 95%, 96%, 97%, 98% or 99% identity thereto.

The inventors found that removing the signal peptide sequence of NiVF improved its synthesis in the cell-free system. Therefore, the NiVF protein may comprise SEQ ID NO: 2, in which the signal peptide sequence is removed, or a sequence having at least 66%, 80%, 81%, 82%, 83%, 84%, 85%, 86%, 87%, 88%, 89%, 90%, 91%, 92%, 93%, 94%, 95%, 96%, 97%, 98% or 99% identity thereto.

The term “identity”, as recognized by those skilled in the art, represents a comparison between two or more amino acid sequences performed using published methods and software known in the art. For example, the compared amino acid sequences are optimally aligned, and the number of amino acid differences are counted and converted to a percentage. For example, if a first amino acid sequence of 50 amino acids is optimally aligned with a second amino acid sequence of 50 amino acids, and 5 out of 50 amino acids differ from the second amino acid sequence, then the first amino acid sequence is said to have 10% identity with the second amino acid sequence.

The nanocarrier may further comprise a cargo molecule. A cargo molecule may comprise any molecule which is to be transported or delivered by a lipid-based nanocarrier. For example, lipid-based nanocarrier cargo may comprise RNA, DNA, active pharmaceutical ingredients, adjuvants, proteins, therapeutic agents, gene editing cargo such as Cas9, etc. Cargo may be organ, tissue or cell type specific.

The cargo molecule is encapsulated within the nanocarrier. As used herein, the term “encapsulate,” “encapsulated,” or “encapsulation” means enclosed or compartmentalized within a membrane. The membrane provides a semi-permeable barrier between the contents encapsulated within the lumen of the membrane and the external environment. Any methods known in the art for encapsulating cargo may be used.

As used herein, “active pharmaceutical ingredient” (“API”) or “small molecule drug” refers to a component that provides pharmacological activity or other direct effect in the diagnosis, cure, mitigation, treatment, or prevention of disease, or to affect the structure or any function of the body of man or animals. As used herein, “therapeutic agent” refers to an agent or compound that relieves to some extent one or more signs, symptoms, or causes of a disease or condition.

The cargo molecule may comprise CpG oligonucleotides. CpGs may be categorized in one of several different classes, e.g. class A, B, C, P, or S CpG. The cargo molecule may comprise two or more different CpGs. The ratio of CpG in the nanocarriers and compositions described herein may be from 10:1 to 1:10, including without limitation 8:1 to 1:8, 6:1 to 1:6, 4:1 to 1:4, 2:1 to 1:2, or approximately 1:1.

In a second aspect, provided herein is a vaccine composition comprising a nanocarrier comprising a membrane; a cell-free protein synthesis system; and at least one of a nucleic acid template encoding a NiVG fusion protein and a nucleic acid template encoding a NiVF fusion protein, wherein the membrane comprises at least one of a first modified lipid and a second modified lipid; and wherein the NiVG fusion protein comprises a first protein tag that conjugates to the first modified lipid and a NiVG Nipah virus protein, and wherein the NiVF fusion protein comprises a second protein tag that conjugates to the second modified lipid and a NiVF Nipah virus protein. The NiVF and NiVG Nipah virus proteins may comprise any of the NiVG and NIVF proteins described herein, including proteins comprising any of SEQ ID NOS: 1-3 or sequences having at least 66%, 80%, 81%, 82%, 83%, 84%, 85%, 86%, 87%, 88%, 89%, 90%, 91%, 92%, 93%, 94%, 95%, 96%, 97%, 98% or 99% identity thereto, and immunogenic fragments thereof.

The term “fusion protein” refers to a protein or polypeptide formed from the combination of two different proteins or protein fragments.

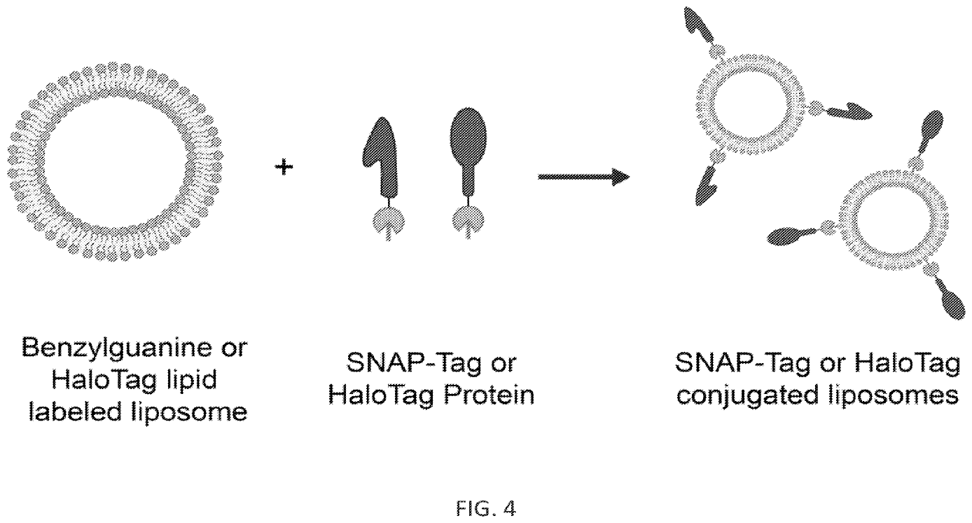

A protein tag may conjugate to a modified lipid. Accordingly, a modified lipid may be used as a substrate to bind a NiVF and NiVG to the nanocarrier surface, as illustrated in FIG. 4.

The protein tag may be a self-labeling enzyme. As used herein, a “self-labeling enzyme” are enzyme tags which catalyze the covalent attachment of an exogenously added synthetic ligand. The synthetic ligands are tag specific and can be coupled to diverse useful labels, such as fluorescent dyes, affinity handles, or solid surfaces. Self-labeling enzymes include, without limitation, SNAP-tag, CLIP-tag, HaloTag, and SpyTag.

The modified lipid may be O⁶-benzylguanine (BG) or BG-derivatives. BG and BG-derivates serve as substrates for SNAP-tag. The modified lipid may be a HaloTag ligand, and therefore serve as a substrate for HaloTag. Other modified lipids may be incorporated into the nanocarrier to avoid detection or the nanocarrier by the immune system, to alter the solubility of the nanocarrier, to alter the bioavailability of the nanocarrier, etc.

SNAP-tag is a 182 residues polypeptide (19.4 kDa) that can be fused to any protein of interest and further specifically and covalently tagged with a suitable ligand, such as a fluorescent dye. HaloTag is a 297 residue (33 kDa) protein derived from a bacterial enzyme, designed to covalently bind to a synthetic ligand. The synthetic ligands may comprise a chloroalkane linker attached to a functional group, e.g. a fluorescent dye or affinity handle.

FIG. 4 illustrates the process of conjugating cell-free expressed viral membrane proteins to liposomes using self-labeling protein tags. Liposomes can be created with BG or HaloTag ligand functionalized lipids, and NiVF and NiVG fusion proteins with SNAP-Tag or HaloTag can be synthesized. When combined, this results in liposomes decorated with the NiVF and NiVG proteins. This process is further described in Peruzzi, J. A., Vu, T. Q., Gunnels, T. F., & Kamat, N. P. (2023). Rapid Generation of Therapeutic Nanoparticles Using Cell-Free Expression Systems. Small Methods, 2201718, and International Application No. PCT/US2023/076459, the contents of which are incorporated by reference herein in their entireties.

The fusion proteins may comprise the same protein tag or different protein tags. Accordingly, the nanocarrier may comprise one or two modified lipids. Any of the nanocarriers described herein may be used.

In a third aspect, provide herein is a vaccine nanoparticle prepared from any of the vaccine compositions described herein, wherein the vaccine nanoparticle is prepared by expressing at least one the nucleic acid templates. An expressed protein comprising a transmembrane domain may integrate with the nanocarrier to form the vaccine nanoparticle. Additionally or alternatively, an expressed protein comprising a protein tag may conjugate with a modified lipid to form the vaccine nanoparticle. In exemplary embodiments, the vaccine nanoparticle is a lipid nanoparticle.

In a fourth aspect, provided herein is a method for eliciting neutralizing antibodies in a subject, the method comprising administering to the subject a therapeutically effective amount of any of the vaccine nanoparticles described herein. In exemplary embodiments, the vaccine nanoparticle is a lipid nanoparticle. A neutralizing antibody is an antibody that defends a cell from a pathogen or infectious particle by neutralizing any effect it has biologically. Neutralization renders the pathogen no longer infectious or pathogenic.

In a fifth aspect, provided herein is a method for treating a Nipah virus infection in a subject, the method comprising administering to the subject a therapeutically effective amount of any of the vaccine nanoparticles described herein.

The term “administration,” as used herein, refers to the introduction of a substance, such as a vaccine, into a subject's body. The administration, e.g., parenteral administration, may include subcutaneous administration, intramuscular administration, transcutaneous administration, intradermal administration, intraperitoneal administration, intraocular administration, intranasal administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The term “immune status” or “immunocompetence,” as used herein, refers to the ability of the body to produce a normal immune response following exposure to an antigen.

Immunocompetence is the opposite of immunodeficiency or immuno-incompetent or immuno-compromised.

The terms “effective amount” or “therapeutically effective amount” refer to an amount sufficient to effect beneficial or desirable biological or clinical results.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. The subject may be a human or other mammal infected with Nipah virus, or at risk of exposure to Nipah virus.

As used herein, “treating” or “treatment” describes the management and care of a subject for the purpose of combating a disease, condition, or disorder. Treating includes the administration of an antibody or composition of the present invention to prevent the onset of the symptoms or complications, to alleviate the symptoms or complications, or to eliminate the disease, condition, or disorder.

In a sixth aspect, provided herein is a method for preparing a vaccine nanoparticle, the method comprising expressing at least one of the nucleic acid templates described herein. The nucleic acid templates are expressed using the cell free systems as described above.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

The following Example is illustrative and should not be interpreted to limit the scope of the claimed subject matter.

Assembly of Vaccines Using Cell-Free Integration of Viral Membrane Proteins Into Liposomes

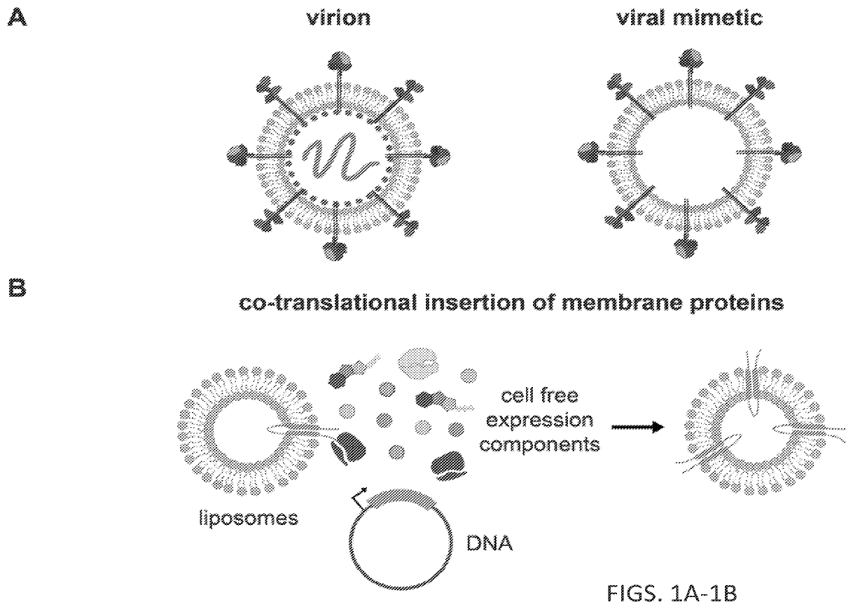

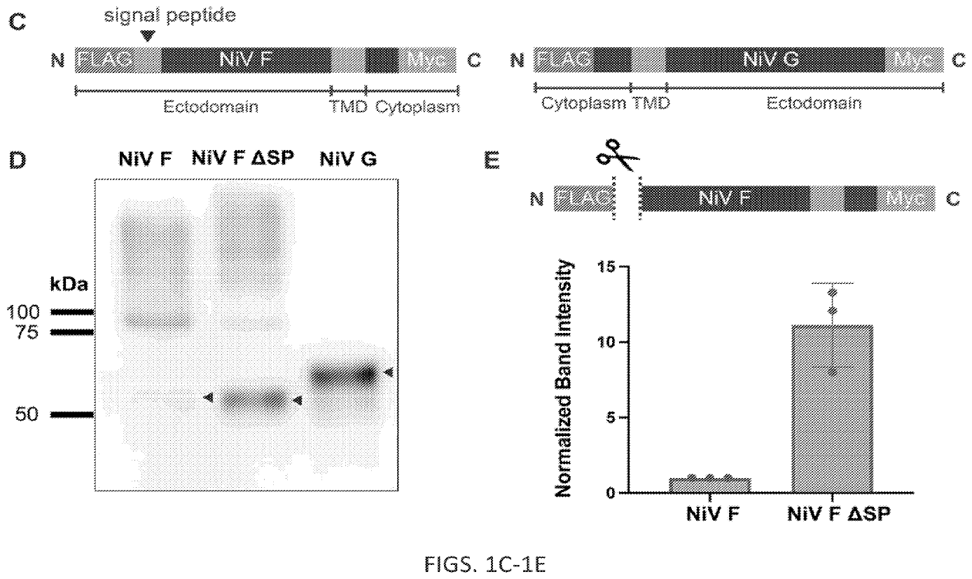

This invention utilizes cell-free protein synthesis systems to integrate viral membrane proteins into liposomes in one-step assembly to generate viral mimetics to serve as vaccines (FIGS. 1A and 1B). Prior to the research surrounding this invention, the design rules for engineering such vaccines for viral diseases were largely unknown. Towards the goal of vaccine development, we have investigated how lipid composition affects the assembly of cell-free viral mimetics (protein integration, orientation, and conformation) using the Nipah virus (NiV) infection as a model and the two immunogenic membrane proteins: glycoprotein (NiV G) and fusion (NiV F) (FIG. 1C).

We first wanted to confirm that NiV F and G proteins could be expressed in liposomes using cell-free protein synthesis (see methods below). We started by expressing NiV F and G proteins in standard PC vesicles and performed western blots on purified liposome samples detecting full-length protein. Here, we found by the single band matching length of the proteins that NiV F and G proteins indeed can be expressed and associated with liposomes (FIG. 1D). To improve expression of type I transmembrane protein NiV F, we deleted the gene sequence encoding the hydrophobic signal peptide (NiV F ΔSP) to improve synthesis of the membrane protein in the environment with liposomes and found that NiV F expression improved by over 8-fold (FIGS. 1D and 1E). NiV F ΔSP was used for subsequent studies.

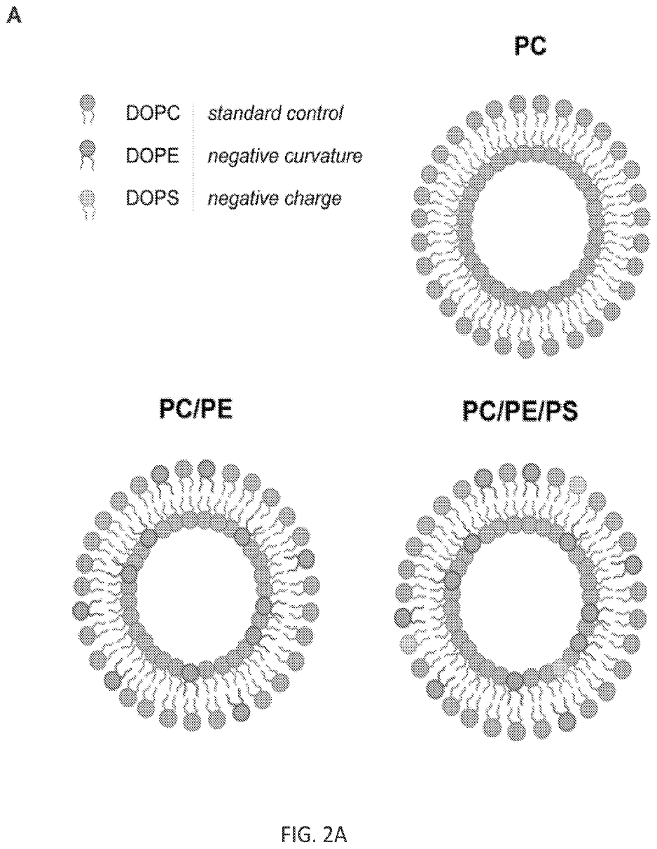

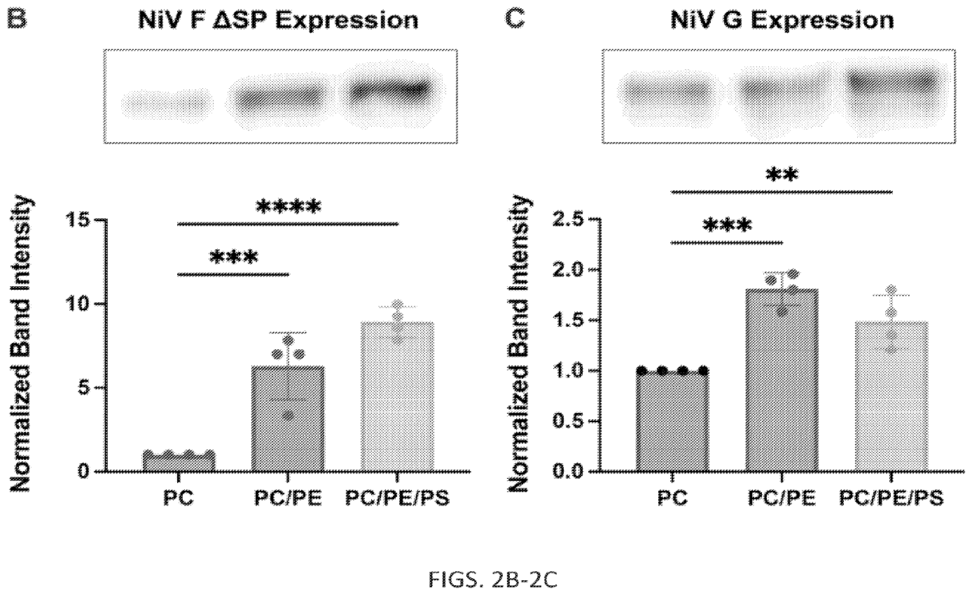

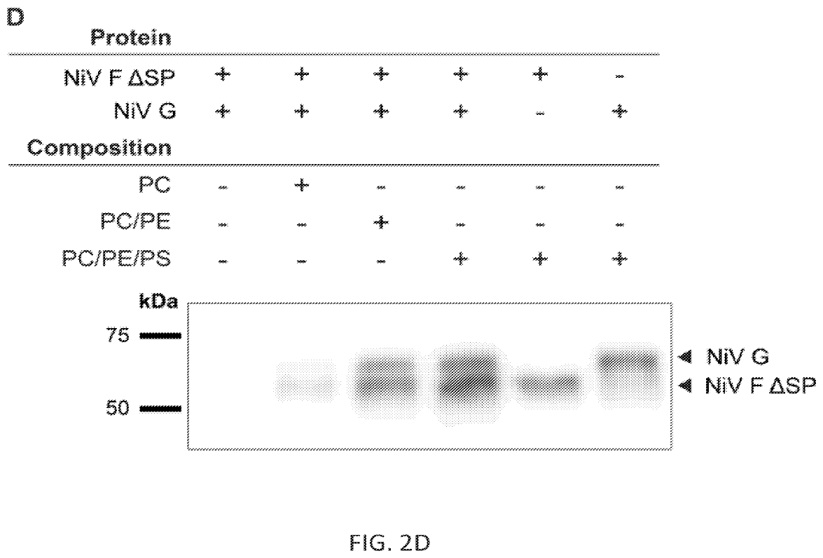

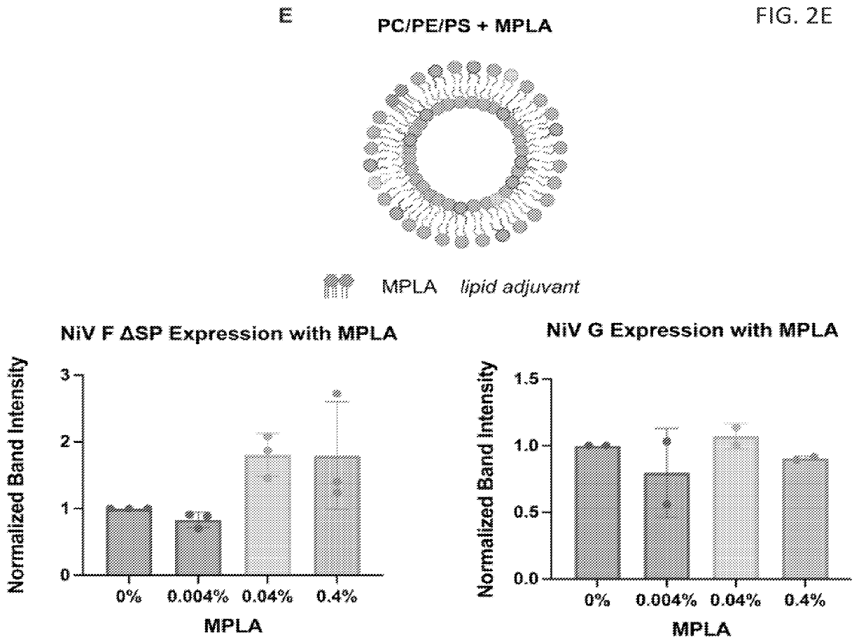

Afterwards, we wanted to explore the effects of altering lipid composition on expression of the Nipah viral membrane proteins. We chose to look at the compositions of PC (standard control), PC/PE (introduce membrane defects to potentially improve cell-free protein integration), and PC/PE/PS (viral mimetic) (FIG. 2A). As shown in the semi-quantitative western blots analyzing full-length NiV F ΔSP or NiV G in the aforementioned compositions, there are significant increases in expression for NiV F ΔSP in PC vs. PC/PE/PS, with a nearly 10-fold increase (FIG. 2B). For NiV G, compositional increases in expression are seen with both PC/PE and PC/PE/PS, with nearly 2-fold and 1.5-fold increases respectively (FIG. 2C).

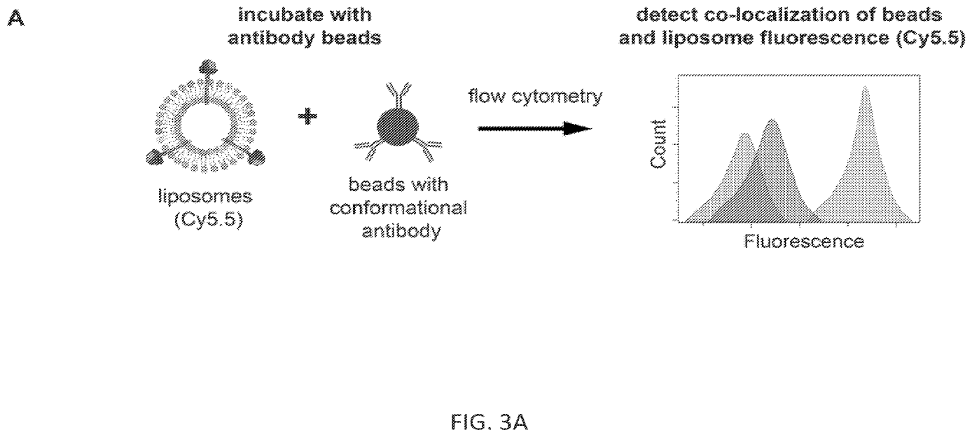

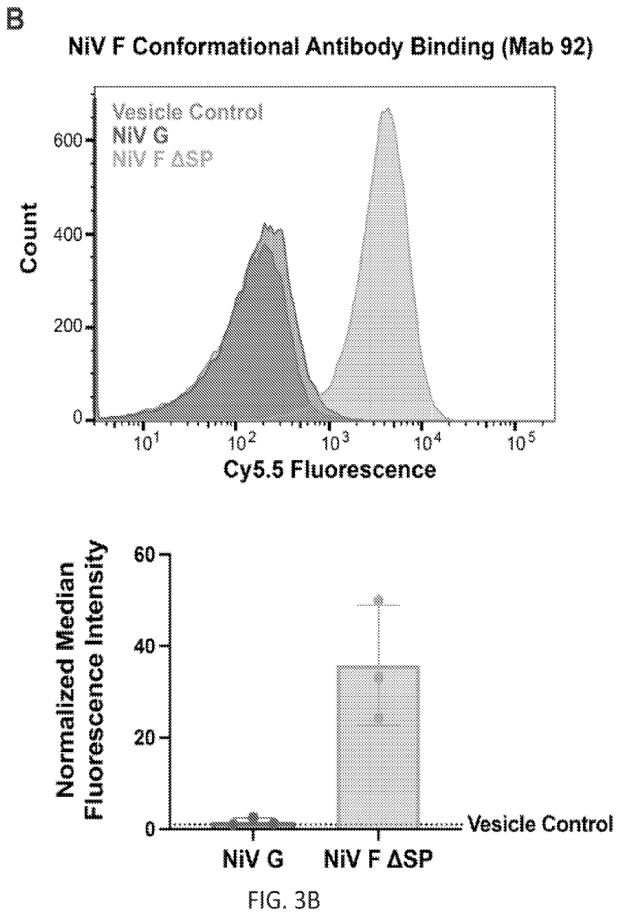

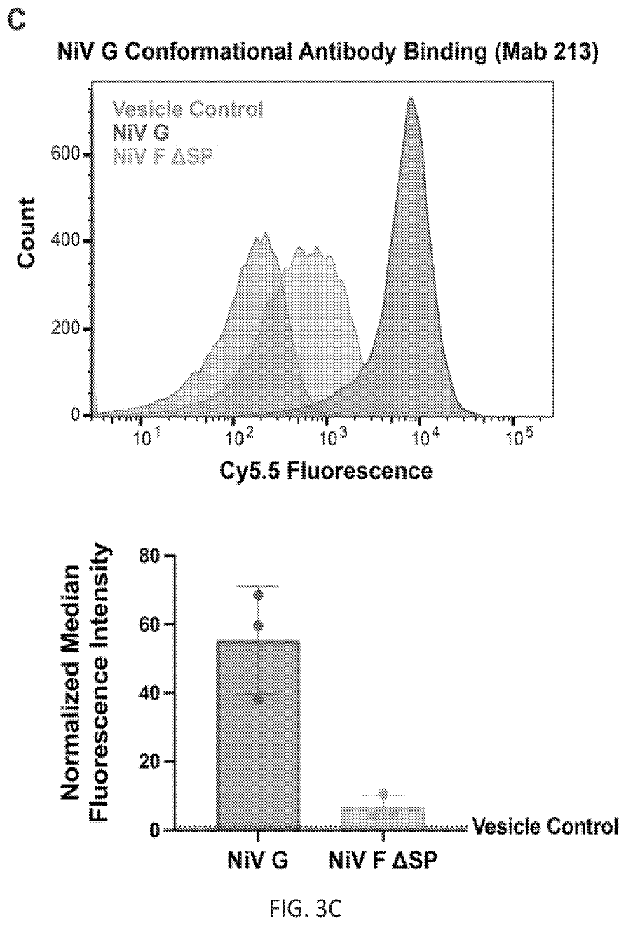

Next, towards the development of a bivalent vaccine, it was demonstrated that both NiV F ΔSP and NiV G proteins can be co-expressed in liposomes and similarly show composition-dependent effects on expression (FIG. 2D). Lastly, using monoclonal antibodies detecting the conformation of viral membrane proteins NiV F and NiV G, we found that not only are the viral membrane proteins expressed and associated with liposomes, but are also folded correctly (FIG. 3). Semi-quantitative western blot analysis reveals that the inclusion of MPLA lipid at amounts of 0%, 0.004%, 0.04%, and 0.4% in PC/PE/PS liposomes does not affect the expression of NiV F ΔSP and NiV G (FIG. 2E).

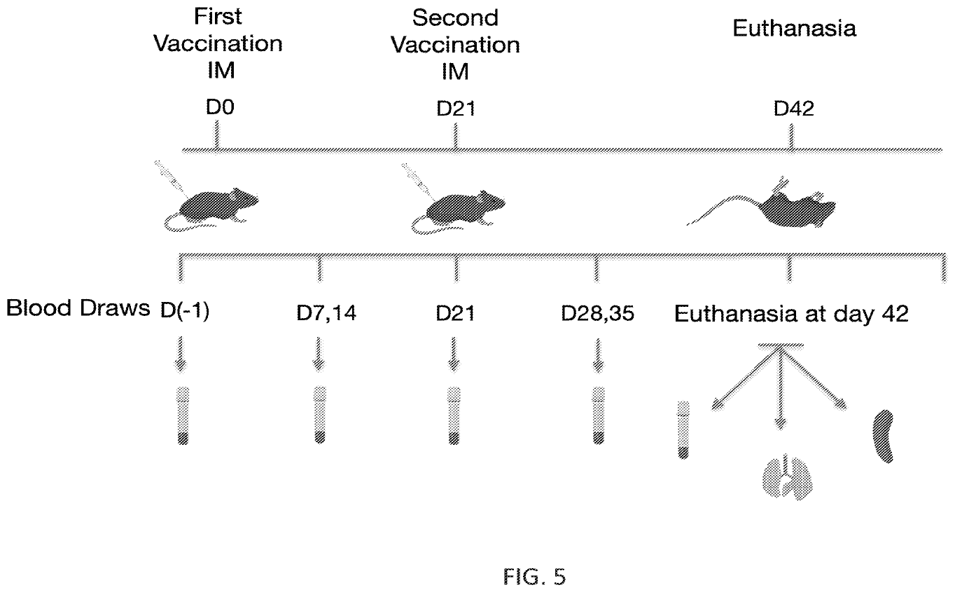

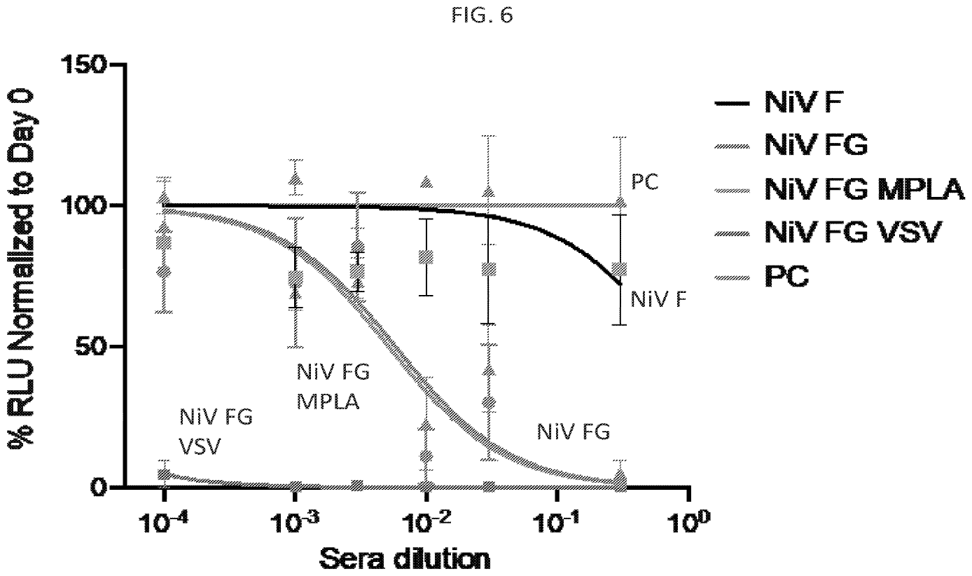

To assess the ability of the NiV F and NiV G nanocarriers, in which the transmembrane domain of NiV F and NiV G were integrated into the liposome membranes, to elicit neutralizing antibodies in vivo, C57BL/6 mice were immunized with the 2 μg of the following 5 vaccines: NiV F liposome vaccine, NiV F and NiV G liposome vaccine in which NiV F and NiV G were co-expressed on one liposome (NiV FG), Niv FG MPLA liposome vaccine, pseudotyped VSV expressing NiV F and NiV G, and control of empty phosphatidylcholine liposome (PC). The vaccination schedule is outlined in FIG. 5. The mice were vaccinated at day 0 and day 21.

A serum neutralization assay was performed. We treated NiV F/G pseudotyped VSV (has Luc gene) with different dilutions of sera, then infected Vero cells. The viral entry was determined based on luminescence produced from the Luc gene and read as relative light units (RLU). The results show that the NiV liposome vaccines elicit neutralizing antibodies in mice, with the NiV FG and NiV FG MPLA vaccines being the most effective cell-free liposomal vaccine formulations.

We have validated that we are able to use cell-free protein synthesis to express and integrate properly folded viral membrane proteins in liposomes and that lipid composition has effects on the expression of such proteins. This opens up applications for expressing other viral membrane proteins through this method of rapid assembly (hours) compared to cell-derived methods to create vaccines for infectious diseases.

### Materials and Methods

Viral mimetics assembly: Codon-optimized NiV F and NiV G constructs were designed for expression in E. coli cell-free protein synthesis systems (New England Biolabs PURExpress®) with N-terminal FLAG and C-terminal Myc tags for western blotting and orientation studies. Liposomes consisted of the phospholipids 1,2-dioleoyl-snglycero-3- with phosphocholine (PC), phosphoethanolamine (PE), and phospho-L-serine (PS) headgroups in PC (100% PC), PC/PE (80% PC, 20% PE), and PC/PE/PS (75% PC, 20% PE, 5% PS) compositions. 18:1 Biotinyl Cap PE and Cy5.5 membrane dye were included for liposome purification and flow cytometry detection. Liposomes were prepared via thin-film hydration and extrusion to 100 nm. NiV F and G constructs were expressed in cell-free protein synthesis system reactions supplemented with 10 mM liposomes. NiV F was optimized for improved expression into liposomes by removing the signal peptide sequence (NiV F ΔSP) and used for remaining studies.

Characterization of viral mimetics: Biotinylated vesicles were purified using magnetic streptavidin beads (Pierce). Western blot analysis of purified proteoliposomes was performed to detect full-length protein production and quantify expression differences between compositions. Protein integration was quantified using semi-quantitative western blots. Integrated protein orientation and structure were validated using flow cytometry via antibody-staining detecting N-terminal tag, C-terminal tag, and native protein conformation (Mab 92 and Mab 213).

TABLE 1
Sequences

| Name | SEQ ID NO: | Sequence |
|---|---|---|
| NIV F | 1 | MDYKDDDDKVVILDKRCYCNLLILILMISECSVGILHYEKL |
| | | SKIGLVKGVTRKYKIKSNPLTKDIVIKMIPNVSNMSQCTGS |
| | | VMENYKTRLNGILTPIKGALEIYKNNTHDLVGDVRLAGVI |
| | | MAGVAIGIATAAQITAGVALYEAMKNADNINKLKSSIEST |
| | | NEAVVKLQETAEKTVYVLTALQDYINTNLVPTIDKISCKQ |
| | | TELSLDLALSKYLSDLLFVFGPNLQDPVSNSMTIQAISQAF |
| | | GGNYETLLRTLGYATEDFDDLLESDSITGQIIYVDLSSYYII |
| | | VRVYFPILTEIQQAYIQELLPVSFNNDNSEWISIVPNFILVRN |
| | | TLISNIEIGFCLITKRSVICNQDYATPMTNNMRECLTGSTEK |
| | | CPRELVVSSHVPRFALSNGVLFANCISVTCQCQTTGRAISQ |
| | | SGEQTLLMIDNTTCPTAVLGNVIISLGKYLGSVNYNSEGIAI |
| | | GPPVFTDKVDISSQISSMNQSLQQSKDYIKEAQRLLDTVNP |
| | | SLISMLSMIILYVLSIASLCIGLITFISFIIVEKKRAAYSRLED |
| | | RRVRPTSSGDLYYIGTEQKLISEEDL |
| NIV F ΔSP | 2 | MDYKDDDDKILHYEKLSKIGLVKGVTRKYKIKSNPLTKDI |
| | | VIKMIPNVSNMSQCTGSVMENYKTRLNGILTPIKGALEIYK |
| | | NNTHDLVGDVRLAGVIMAGVAIGIATAAQITAGVALYEA |
| | | MKNADNINKLKSSIESTNEAVVKLQETAEKTVYVLTALQD |
| | | YINTNLVPTIDKISCKQTELSLDLALSKYLSDLLFVFGPNLQ |
| | | DPVSNSMTIQAISQAFGGNYETLLRTLGYATEDFDDLLESD |
| | | SITGQIIYVDLSSYYIIVRVYFPILTEIQQAYIQELLPVSFNND |
| | | NSEWISIVPNFILVRNTLISNIEIGFCLITKRSVICNQDYATP |
| | | MTNNMRECLTGSTEKCPRELVVSSHVPRFALSNGVLFANC |
| | | ISVTCQCQTTGRAISQSGEQTLLMIDNTTCPTAVLGNVIISL |
| | | GKYLGSVNYNSEGIAIGPPVFTDKVDISSQISSMNQSLQQS |
| | | KDYIKEAQRLLDTVNPSLISMLSMIILYVLSIASLCIGLITFIS |
| | | FIIVEKKRAAYSRLEDRRVRPTSSGDLYYIGTEQKLISEEDL |
| NIVG | 3 | MDYKDDDDKPAENKKVRFENTTSDKGKIPSKVIKSYYGT |
| | | MDIKKINEGLLDSKILSAFNTVIALLGSIVIIVMNIMIIQNYT |
| | | RSTDNQAVIKDALQGIQQQIKGLADKIGTEIGPKVSLIDTSS |
| | | TITIPANIGLLGSKISQSTASINENVNEKCKFTLPPLKIHECNI |
| | | SCPNPLPFREYRPQTEGVSNLVGLPNNICLQKTSNQILKPK |
| | | LISYTLPVVGQSGTCITDPLLAMDEGYFAYSHLERIGSCSR |
| | | GVSKQRIIGVGEVLDRGDEVPSLFMTNVWTPPNPNTVYHC |
| | | SAVYNNEFYYVLCAVSTVGDPILNSTYWSGSLMMTRLAV |
| | | KPKSNGGGYNQHQLALRSIEKGRYDKVMPYGPSGIKQGD |
| | | TLYFPAVGFLVRTEFKYNDSNCPITKCQYSKPENCRLSMGI |
| | | RPNSHYILRSGLLKYNLSDGENPKVVFIEISDQRLSIGSPSKI |
| | | YDSLGQPVFYQASFSWDTMIKFGDVLTVNPLVVNWRNNT |
| | | VISRPGQSQCPRFNTCPEICWEGVYNDAFLIDRINWISAGV |
| | | FLDSNQTAENPVFTVFKDNEILYRAQLASEDTNAQKTITN |
| | | CFLLKNKIWCISLVEIYDTGDNVIRPKLFAVKIPEQCTEQKL |
| | | ISEEDL |
| NIV F ΔASP | 4 | MDYKDDDDKILHYEKLSKIGLVKGVTRKYKIKSNPLTKDI |
| SNAP-tag | | VIKMIPNVSNMSQCTGSVMENYKTRLNGILTPIKGALEIYK |
| | | NNTHDLVGDVRLAGVIMAGVAIGIATAAQITAGVALYEA |
| | | MKNADNINKLKSSIESTNEAVVKLQETAEKTVYVLTALQD |
| | | YINTNLVPTIDKISCKQTELSLDLALSKYLSDLLFVFGPNLQ |
| | | DPVSNSMTIQAISQAFGGNYETLLRTLGYATEDFDDLLESD |
| | | SITGQIIYVDLSSYYIIVRVYFPILTEIQQAYIQELLPVSENND |
| | | NSEWISIVPNFILVRNTLISNIEIGFCLITKRSVICNQDYATP |
| | | MTNNMRECLTGSTEKCPRELVVSSHVPRFALSNGVLFANC |
| | | ISVTCQCQTTGRAISQSGEQTLLMIDNTTCPTAVLGNVIISL |
| | | GKYLGSVNYNSEGIAIGPPVFTDKVDISSQISSMNQSLQQS |
| | | KDYIKEAQRLLDTVNPSLISMLSMIILGSSGASPAAPAPASP |
| | | AAPAPSAPAGGMDKDCEMKRTTLDSPLGKLELSGCEQGL |
| | | HEIKLLGKGTSAADAVEVPAPAAVLGGPEPLMQATAWLN |
| | | AYFHQPEAIEEFPVPALHHPVFQQESFTRQVLWKLLKVVK |
| | | FGEVISYQQLAALAGNPAATAAVKTALSGNPVPILIPCHRV |
| | | VSSSGAVGGYEGGLAVKEWLLAHEGHRLGKPGLGHMGS |
| | | EQKLISEEDL |
| NIV F ΔSP | 5 | MDYKDDDDKILHYEKLSKIGLVKGVTRKYKIKSNPLTKDI |
| HaloTag | | VIKMIPNVSNMSQCTGSVMENYKTRLNGILTPIKGALEIYK |
| | | NNTHDLVGDVRLAGVIMAGVAIGIATAAQITAGVALYEA |
| | | MKNADNINKLKSSIESTNEAVVKLQETAEKTVYVLTALQD |
| | | YINTNLVPTIDKISCKQTELSLDLALSKYLSDLLFVFGPNLQ |
| | | DPVSNSMTIQAISQAFGGNYETLLRTLGYATEDFDDLLESD |
| | | SITGQIIYVDLSSYYIIVRVYFPILTEIQQAYIQELLPVSFNND |
| | | NSEWISIVPNFILVRNTLISNIEIGFCLITKRSVICNQDYATP |
| | | MTNNMRECLTGSTEKCPRELVVSSHVPRFALSNGVLFANC |
| | | ISVTCQCQTTGRAISQSGEQTLLMIDNTTCPTAVLGNVIISL |
| | | GKYLGSVNYNSEGIAIGPPVFTDKVDISSQISSMNQSLQQS |
| | | KDYIKEAQRLLDTVNPSLISMLSMIILGSSGASPAAPAPASP |
| | | AAPAPSAPAGGMAEIGTGFPFDPHYVEVLGERMHYVDVG |
| | | PRDGTPVLFLHGNPTSSYVWRNIIPHVAPTHRCIAPDLIGM |
| | | GKSDKPDLGYFFDDHVRFMDAFIEALGLEEVVLVIHDWG |
| | | SALGFHWAKRNPERVKGIAFMEFIRPIPTWDEWPEFARET |
| | | FQAFRTTDVGRKLIIDQNVFIEGTLPMGVVRPLTEVEMDH |
| | | YREPFLNPVDREPLWRFPNELPIAGEPANIVALVEEYMDW |
| | | LHQSPVPKLLFWGTPGVLIPPAEAARLAKSLPNCKAVDIGP |
| | | GLNLLQEDNPDLIGSEIARWLSTLEISGHMGSEQKLISEEDL |
| NIV G SNAP- | 6 | MDYKDDDDKHMGSMDKDCEMKRTTLDSPLGKLELSGCE |
| tag | | QGLHEIKLLGKGTSAADAVEVPAPAAVLGGPEPLMQATA |
| | | WLNAYFHQPEAIEEFPVPALHHPVFQQESFTRQVLWKLLK |
| | | VVKFGEVISYQQLAALAGNPAATAAVKTALSGNPVPILIPC |
| | | HRVVSSSGAVGGYEGGLAVKEWLLAHEGHRLGKPGLGG |
| | | SSGASPAAPAPASPAAPAPSAPAGGQNYTRSTDNQAVIKD |
| | | ALQGIQQQIKGLADKIGTEIGPKVSLIDTSSTITIPANIGLLG |
| | | SKISQSTASINENVNEKCKFTLPPLKIHECNISCPNPLPFREY |
| | | RPQTEGVSNLVGLPNNICLQKTSNQILKPKLISYTLPVVGQ |
| | | SGTCITDPLLAMDEGYFAYSHLERIGSCSRGVSKQRIIGVG |
| | | EVLDRGDEVPSLFMTNVWTPPNPNTVYHCSAVYNNEFYY |
| | | VLCAVSTVGDPILNSTYWSGSLMMTRLAVKPKSNGGGYN |
| | | QHQLALRSIEKGRYDKVMPYGPSGIKQGDTLYFPAVGFLV |
| | | RTEFKYNDSNCPITKCQYSKPENCRLSMGIRPNSHYILRSG |
| | | LLKYNLSDGENPKVVFIEISDQRLSIGSPSKIYDSLGQPVFY |
| | | QASFSWDTMIKFGDVLTVNPLVVNWRNNTVISRPGQSQC |
| | | PRFNTCPEICWEGVYNDAFLIDRINWISAGVFLDSNQTAEN |
| | | PVFTVFKDNEILYRAQLASEDTNAQKTITNCFLLKNKIWCI |
| | | SLVEIYDTGDNVIRPKLFAVKIPEQCTEQKLISEEDL |
| NiV G HaloTag | 7 | MDYKDDDDKHMGSMAEIGTGFPFDPHYVEVLGERMHYV |
| | | DVGPRDGTPVLFLHGNPTSSYVWRNIIPHVAPTHRCIAPDL |
| | | IGMGKSDKPDLGYFFDDHVRFMDAFIEALGLEEVVLVIHD |
| | | WGSALGFHWAKRNPERVKGIAFMEFIRPIPTWDEWPEFAR |
| | | ETFQAFRTTDVGRKLIIDQNVFIEGTLPMGVVRPLTEVEMD |
| | | HYREPFLNPVDREPLWRFPNELPIAGEPANIVALVEEYMD |
| | | WLHQSPVPKLLFWGTPGVLIPPAEAARLAKSLPNCKAVDI |
| | | GPGLNLLQEDNPDLIGSEIARWLSTLEISGGSSGASPAAPAP |
| | | ASPAAPAPSAPAGGQNYTRSTDNQAVIKDALQGIQQQIKG |
| | | LADKIGTEIGPKVSLIDTSSTITIPANIGLLGSKISQSTASINE |
| | | NVNEKCKFTLPPLKIHECNISCPNPLPFREYRPQTEGVSNL |
| | | VGLPNNICLQKTSNQILKPKLISYTLPVVGQSGTCITDPLLA |
| | | MDEGYFAYSHLERIGSCSRGVSKQRIIGVGEVLDRGDEVP |
| | | SLFMTNVWTPPNPNTVYHCSAVYNNEFYYVLCAVSTVGD |
| | | PILNSTYWSGSLMMTRLAVKPKSNGGGYNQHQLALRSIE |
| | | KGRYDKVMPYGPSGIKQGDTLYFPAVGFLVRTEFKYNDS |
| | | NCPITKCQYSKPENCRLSMGIRPNSHYILRSGLLKYNLSDG |
| | | ENPKVVFIEISDQRLSIGSPSKIYDSLGQPVFYQASFSWDTM |
| | | IKFGDVLTVNPLVVNWRNNTVISRPGQSQCPRFNTCPEIC |
| | | WEGVYNDAFLIDRINWISAGVFLDSNQTAENPVFTVFKDN |
| | | EILYRAQLASEDTNAQKTITNCFLLKNKIWCISLVEIYDTG |
| | | DNVIRPKLFAVKIPEQCTEQKLISEEDL |
| NIV F | 8 | ATGGACTATAAAGACGATGACGACAAAGTGGTGATTCT |
| | | GGATAAACGTTGTTATTGCAATCTGCTGATTCTGATCCT |
| | | GATGATTAGCGAATGTAGCGTTGGTATCCTGCACTATG |
| | | AAAAACTGAGCAAAATTGGTCTGGTTAAAGGTGTGACC |
| | | CGCAAATACAAAATCAAAAGCAATCCGCTGACCAAGGA |
| | | CATCGTGATCAAAATGATTCCGAATGTGAGCAATATGA |
| | | GCCAGTGTACCGGTAGCGTTATGGAAAACTATAAAACC |
| | | CGTCTGAATGGTATTCTGACCCCGATTAAAGGTGCACTG |
| | | GAAATCTATAAGAACAACACCCATGATCTGGTTGGTGA |
| | | TGTTCGTCTGGCAGGCGTTATTATGGCTGGTGTTGCAAT |
| | | TGGTATTGCAACCGCAGCACAGATTACCGCAGGCGTTG |
| | | CACTGTATGAAGCAATGAAAAATGCCGACAACATCAAC |
| | | AAACTGAAAAGCAGCATTGAAAGCACCAATGAAGCAG |
| | | TTGTTAAACTGCAAGAAACCGCAGAAAAAACCGTTTAT |
| | | GTTCTGACCGCACTGCAGGATTACATTAATACCAATCTG |
| | | GTTCCGACCATCGATAAAATCAGCTGTAAACAGACCGA |
| | | ACTGAGCCTGGATCTGGCCCTGAGCAAATATCTGAGCG |
| | | ATCTGCTGTTTGTTTTTGGTCCGAATCTGCAGGATCCGG |
| | | TTAGCAATAGCATGACCATTCAGGCAATTAGCCAGGCA |
| | | TTTGGTGGTAATTATGAAACCCTGCTGCGTACCCTGGGT |
| | | TATGCAACCGAAGATTTTGATGATCTGCTGGAAAGCGA |
| | | TAGCATTACCGGTCAGATTATCTATGTTGATCTGAGCAG |
| | | CTACTATATTATCGTGCGTGTGTATTTTCCGATCCTGAC |
| | | CGAAATTCAGCAGGCATATATCCAAGAACTGCTGCCGG |
| | | TTAGCTTTAATAACGATAATAGCGAATGGATTAGCATC |
| | | GTGCCGAACTTTATTCTGGTTCGTAATACCCTGATTAGC |
| | | AACATCGAAATTGGCTTTTGCCTGATTACCAAACGTAGC |
| | | GTGATTTGCAATCAGGATTATGCGACCCCGATGACCAA |
| | | TAATATGCGTGAATGTCTGACAGGTAGCACCGAAAAAT |
| | | GTCCGCGTGAACTGGTTGTTAGCAGCCATGTTCCGCGTT |
| | | TTGCACTGAGCAATGGTGTTCTGTTTGCAAATTGTATTA |
| | | GCGTTACCTGCCAGTGTCAGACCACCGGTCGTGCCATTA |
| | | GCCAGAGCGGTGAACAGACCCTGCTGATGATTGATAAT |
| | | ACCACCTGTCCGACCGCAGTTCTGGGTAATGTGATTATT |
| | | AGCCTGGGTAAATATCTGGGCAGCGTGAATTATAACAG |
| | | CGAAGGTATTGCCATTGGTCCGCCTGTTTTTACCGATAA |
| | | AGTTGATATTAGCTCCCAGATCAGCAGCATGAATCAGA |
| | | GCCTGCAGCAGAGCAAAGATTATATCAAAGAAGCACAG |
| | | CGTCTGCTGGATACCGTTAATCCGAGCCTGATTAGTATG |
| | | CTGAGCATGATTATTCTGTACGTTCTGAGCATTGCAAGC |
| | | CTGTGTATTGGTCTGATTACCTTTATCAGCTTCATCATC |
| | | GTGGAAAAAAAACGTGCAGCATATAGCCGTCTGGAAGA |
| | | TCGTCGTGTTCGTCCGACCAGCAGCGGTGATCTGTATTA |
| | | TATCGGCACCGAACAGAAACTGATCAGCGAAGAAGATC |
| | | TGTAA |
| NIV F ΔSP | 9 | ATGGACTATAAAGACGATGACGACAAAATCCTGCACTA |
| | | TGAAAAACTGAGCAAAATTGGTCTGGTTAAAGGTGTGA |
| | | CCCGCAAATACAAAATCAAAAGCAATCCGCTGACCAAG |
| | | GACATCGTGATCAAAATGATTCCGAATGTGAGCAATAT |
| | | GAGCCAGTGTACCGGTAGCGTTATGGAAAACTATAAAA |
| | | CCCGTCTGAATGGTATTCTGACCCCGATTAAAGGTGCAC |
| | | TGGAAATCTATAAGAACAACACCCATGATCTGGTTGGT |
| | | GATGTTCGTCTGGCAGGCGTTATTATGGCTGGTGTTGCA |
| | | ATTGGTATTGCAACCGCAGCACAGATTACCGCAGGCGT |
| | | TGCACTGTATGAAGCAATGAAAAATGCCGACAACATCA |
| | | ACAAACTGAAAAGCAGCATTGAAAGCACCAATGAAGC |
| | | AGTTGTTAAACTGCAAGAAACCGCAGAAAAAACCGTTT |
| | | ATGTTCTGACCGCACTGCAGGATTACATTAATACCAATC |
| | | TGGTTCCGACCATCGATAAAATCAGCTGTAAACAGACC |
| | | GAACTGAGCCTGGATCTGGCCCTGAGCAAATATCTGAG |
| | | CGATCTGCTGTTTGTTTTTGGTCCGAATCTGCAGGATCC |
| | | GGTTAGCAATAGCATGACCATTCAGGCAATTAGCCAGG |
| | | CATTTGGTGGTAATTATGAAACCCTGCTGCGTACCCTGG |
| | | GTTATGCAACCGAAGATTTTGATGATCTGCTGGAAAGC |
| | | GATAGCATTACCGGTCAGATTATCTATGTTGATCTGAGC |
| | | AGCTACTATATTATCGTGCGTGTGTATTTTCCGATCCTG |
| | | ACCGAAATTCAGCAGGCATATATCCAAGAACTGCTGCC |
| | | GGTTAGCTTTAATAACGATAATAGCGAATGGATTAGCA |
| | | TCGTGCCGAACTTTATTCTGGTTCGTAATACCCTGATTA |
| | | GCAACATCGAAATTGGCTTTTGCCTGATTACCAAACGTA |
| | | GCGTGATTTGCAATCAGGATTATGCGACCCCGATGACC |
| | | AATAATATGCGTGAATGTCTGACAGGTAGCACCGAAAA |
| | | ATGTCCGCGTGAACTGGTTGTTAGCAGCCATGTTCCGCG |
| | | TTTTGCACTGAGCAATGGTGTTCTGTTTGCAAATTGTAT |
| | | TAGCGTTACCTGCCAGTGTCAGACCACCGGTCGTGCCAT |
| | | TAGCCAGAGCGGTGAACAGACCCTGCTGATGATTGATA |
| | | ATACCACCTGTCCGACCGCAGTTCTGGGTAATGTGATTA |
| | | TTAGCCTGGGTAAATATCTGGGCAGCGTGAATTATAAC |
| | | AGCGAAGGTATTGCCATTGGTCCGCCTGTTTTTACCGAT |
| | | AAAGTTGATATTAGCTCCCAGATCAGCAGCATGAATCA |
| | | GAGCCTGCAGCAGAGCAAAGATTATATCAAAGAAGCAC |
| | | AGCGTCTGCTGGATACCGTTAATCCGAGCCTGATTAGTA |
| | | TGCTGAGCATGATTATTCTGTACGTTCTGAGCATTGCAA |
| | | GCCTGTGTATTGGTCTGATTACCTTTATCAGCTTCATCA |
| | | TCGTGGAAAAAAAACGTGCAGCATATAGCCGTCTGGAA |
| | | GATCGTCGTGTTCGTCCGACCAGCAGCGGTGATCTGTAT |
| | | TATATCGGCACCGAACAGAAACTGATCAGCGAAGAAGA |
| | | TCTGTAA |
| NIVG | 10 | ATGGACTATAAAGACGATGACGACAAACCGGCCGAGA |
| | | ACAAGAAGGTCCGCTTTGAAAATACGACTAGTGATAAG |
| | | GGTAAGATCCCTTCGAAAGTAATCAAGAGCTACTACGG |
| | | TACCATGGATATAAAGAAGATAAACGAGGGACTGTTAG |
| | | ACTCAAAGATACTGTCTGCCTTCAACACAGTGATCGCGT |
| | | TACTGGGGTCTATTGTCATTATAGTAATGAACATAATGA |
| | | TAATACAAAATTACACCCGCTCGACAGATAACCAGGCA |
| | | GTAATCAAGGACGCGTTACAAGGTATTCAACAACAAAT |
| | | CAAAGGTCTTGCCGACAAGATTGGCACCGAAATCGGTC |
| | | CAAAGGTCAGCCTCATTGACACATCGAGTACCATAACC |
| | | ATCCCAGCTAACATCGGCCTTCTGGGCTCTAAAATTTCC |
| | | CAGAGTACAGCCTCTATCAACGAGAACGTAAACGAGAA |
| | | ATGCAAGTTTACTTTGCCCCCGCTGAAAATTCACGAATG |
| | | CAACATTAGTTGTCCGAACCCTTTGCCATTTCGAGAGTA |
| | | CCGCCCCCAAACAGAGGGCGTGTCAAACTTGGTAGGAC |
| | | TCCCCAATAATATATGTCTCCAGAAGACTTCAAATCAA |
| | | ATACTGAAGCCTAAGTTGATATCATACACCCTGCCGGTC |
| | | GTCGGCCAAAGCGGCACATGTATAACAGACCCGCTCCT |
| | | TGCTATGGACGAGGGTTATTTTGCCTACAGCCACCTGGA |
| | | GCGCATCGGGTCATGTAGCCGTGGCGTCTCTAAGCAGC |
| | | GTATTATTGGCGTTGGGGAAGTCTTAGACCGTGGAGAC |
| | | GAGGTTCCTAGTTTATTCATGACCAATGTGTGGACACCC |
| | | CCAAACCCCAATACGGTCTATCACTGTTCTGCAGTATAC |
| | | AACAACGAATTTTACTACGTGCTTTGTGCAGTATCAACC |
| | | GTAGGCGATCCGATTCTCAACTCCACGTATTGGAGTGG |
| | | AAGCTTGATGATGACGAGACTTGCTGTTAAGCCAAAGT |
| | | CGAATGGCGGTGGCTACAATCAACATCAATTAGCGCTC |
| | | CGGAGTATTGAGAAGGGTCGCTACGACAAAGTTATGCC |
| | | CTACGGCCCTAGTGGTATAAAGCAAGGGGACACACTGT |
| | | ACTTCCCTGCAGTTGGTTTTCTGGTGCGTACCGAATTCA |
| | | AATACAACGACTCGAACTGCCCGATAACTAAATGCCAG |
| | | TACTCCAAGCCTGAAAATTGCCGCCTCTCGATGGGCAT |
| | | ACGCCCTAATTCTCACTACATTTTGAGATCAGGGCTGCT |
| | | TAAGTACAATCTGAGTGACGGCGAAAACCCCAAGGTGG |
| | | TGTTTATCGAGATATCTGACCAAAGACTCTCCATCGGTA |
| | | GCCCTTCCAAAATCTATGACTCACTTGGTCAGCCCGTCT |
| | | TCTATCAAGCTTCATTCAGTTGGGACACAATGATTAAGT |
| | | TCGGAGACGTTCTGACGGTAAACCCTCTGGTCGTGAAC |
| | | TGGCGCAATAACACCGTTATAAGCCGTCCAGGACAATC |
| | | GCAGTGTCCTCGTTTCAACACCTGCCCAGAGATCTGCTG |
| | | GGAAGGCGTCTACAACGACGCGTTTCTGATCGATCGGA |
| | | TTAACTGGATAAGTGCAGGGGTTTTCTTGGACAGCAAT |
| | | CAAACCGCCGAAAACCCTGTTTTCACAGTGTTCAAGGA |
| | | CAACGAAATATTGTACCGCGCCCAACTTGCTTCTGAGG |
| | | ACACGAACGCACAGAAAACCATCACAAATTGCTTTCTG |
| | | TTGAAGAATAAGATTTGGTGTATAAGCCTTGTGGAAAT |
| | | CTACGACACTGGTGACAACGTCATAAGACCGAAGTTAT |
| | | TTGCAGTGAAGATCCCGGAGCAATGCACAGAACAGAAA |
| | | CTGATCAGCGAAGAAGATCTGTAA |
| NIV F ΔSP | 11 | ATGGACTATAAAGACGATGACGACAAAATCCTGCACTA |
| SNAP-tag | | TGAAAAACTGAGCAAAATTGGTCTGGTTAAAGGTGTGA |
| | | CCCGCAAATACAAAATCAAAAGCAATCCGCTGACCAAG |
| | | GACATCGTGATCAAAATGATTCCGAATGTGAGCAATAT |
| | | GAGCCAGTGTACCGGTAGCGTTATGGAAAACTATAAAA |
| | | CCCGTCTGAATGGTATTCTGACCCCGATTAAAGGTGCAC |
| | | TGGAAATCTATAAGAACAACACCCATGATCTGGTTGGT |
| | | GATGTTCGTCTGGCAGGCGTTATTATGGCTGGTGTTGCA |
| | | ATTGGTATTGCAACCGCAGCACAGATTACCGCAGGCGT |
| | | TGCACTGTATGAAGCAATGAAAAATGCCGACAACATCA |
| | | ACAAACTGAAAAGCAGCATTGAAAGCACCAATGAAGC |
| | | AGTTGTTAAACTGCAAGAAACCGCAGAAAAAACCGTTT |
| | | ATGTTCTGACCGCACTGCAGGATTACATTAATACCAATC |
| | | TGGTTCCGACCATCGATAAAATCAGCTGTAAACAGACC |
| | | GAACTGAGCCTGGATCTGGCCCTGAGCAAATATCTGAG |
| | | CGATCTGCTGTTTGTTTTTGGTCCGAATCTGCAGGATCC |
| | | GGTTAGCAATAGCATGACCATTCAGGCAATTAGCCAGG |
| | | CATTTGGTGGTAATTATGAAACCCTGCTGCGTACCCTGG |
| | | GTTATGCAACCGAAGATTTTGATGATCTGCTGGAAAGC |
| | | GATAGCATTACCGGTCAGATTATCTATGTTGATCTGAGC |
| | | AGCTACTATATTATCGTGCGTGTGTATTTTCCGATCCTG |
| | | ACCGAAATTCAGCAGGCATATATCCAAGAACTGCTGCC |
| | | GGTTAGCTTTAATAACGATAATAGCGAATGGATTAGCA |
| | | TCGTGCCGAACTTTATTCTGGTTCGTAATACCCTGATTA |
| | | GCAACATCGAAATTGGCTTTTGCCTGATTACCAAACGTA |
| | | GCGTGATTTGCAATCAGGATTATGCGACCCCGATGACC |
| | | AATAATATGCGTGAATGTCTGACAGGTAGCACCGAAAA |
| | | ATGTCCGCGTGAACTGGTTGTTAGCAGCCATGTTCCGCG |
| | | TTTTGCACTGAGCAATGGTGTTCTGTTTGCAAATTGTAT |
| | | TAGCGTTACCTGCCAGTGTCAGACCACCGGTCGTGCCAT |
| | | TAGCCAGAGCGGTGAACAGACCCTGCTGATGATTGATA |
| | | ATACCACCTGTCCGACCGCAGTTCTGGGTAATGTGATTA |
| | | TTAGCCTGGGTAAATATCTGGGCAGCGTGAATTATAAC |
| | | AGCGAAGGTATTGCCATTGGTCCGCCTGTTTTTACCGAT |
| | | AAAGTTGATATTAGCTCCCAGATCAGCAGCATGAATCA |
| | | GAGCCTGCAGCAGAGCAAAGATTATATCAAAGAAGCAC |
| | | AGCGTCTGCTGGATACCGTTAATCCGAGCCTGATTAGTA |
| | | TGCTGAGCATGATTATTCTGGGTAGCTCAGGAGCAAGT |
| | | CCGGCAGCACCGGCACCGGCATCACCAGCAGCTCCAGC |
| | | ACCTAGTGCACCGGCAGGCGGTATGGACAAAGATTGCG |
| | | AAATGAAACGTACCACCCTGGATAGCCCGCTGGGCAAA |
| | | CTGGAACTGAGCGGCTGCGAACAGGGCCTGCATGAAAT |
| | | TAAACTGCTGGGTAAAGGCACCAGCGCGGCCGATGCGG |
| | | TTGAAGTTCCGGCCCCGGCCGCCGTGCTGGGTGGTCCG |
| | | GAACCGCTGATGCAGGCGACCGCGTGGCTGAACGCGTA |
| | | TTTTCATCAGCCGGAAGCGATTGAAGAATTTCCGGTTCC |
| | | GGCGCTGCATCATCCGGTGTTTCAGCAGGAGAGCTTTA |
| | | CCCGTCAGGTGCTGTGGAAACTGCTGAAAGTGGTTAAA |
| | | TTTGGCGAAGTGATTAGCTATCAGCAGCTGGCGGCCCT |
| | | GGCGGGTAATCCGGCGGCCACCGCCGCCGTTAAAACCG |
| | | CGCTGAGCGGTAACCCGGTGCCGATTCTGATTCCGTGCC |
| | | ATCGTGTGGTTAGCTCTAGCGGTGCGGTTGGCGGTTATG |
| | | AAGGTGGTCTGGCGGTGAAAGAGTGGCTGCTGGCCCAT |
| | | GAAGGTCATCGTCTGGGTAAACCGGGTCTGGGACACAT |
| | | GGGTAGCGAACAGAAACTGATCAGCGAAGAAGATCTGT |
| | | AA |
| NIV F ΔSP | 12 | ATGGACTATAAAGACGATGACGACAAAATCCTGCACTA |
| HaloTag | | TGAAAAACTGAGCAAAATTGGTCTGGTTAAAGGTGTGA |
| | | CCCGCAAATACAAAATCAAAAGCAATCCGCTGACCAAG |
| | | GACATCGTGATCAAAATGATTCCGAATGTGAGCAATAT |
| | | GAGCCAGTGTACCGGTAGCGTTATGGAAAACTATAAAA |
| | | CCCGTCTGAATGGTATTCTGACCCCGATTAAAGGTGCAC |
| | | TGGAAATCTATAAGAACAACACCCATGATCTGGTTGGT |
| | | GATGTTCGTCTGGCAGGCGTTATTATGGCTGGTGTTGCA |
| | | ATTGGTATTGCAACCGCAGCACAGATTACCGCAGGCGT |
| | | TGCACTGTATGAAGCAATGAAAAATGCCGACAACATCA |
| | | ACAAACTGAAAAGCAGCATTGAAAGCACCAATGAAGC |
| | | AGTTGTTAAACTGCAAGAAACCGCAGAAAAAACCGTTT |
| | | ATGTTCTGACCGCACTGCAGGATTACATTAATACCAATC |
| | | TGGTTCCGACCATCGATAAAATCAGCTGTAAACAGACC |
| | | GAACTGAGCCTGGATCTGGCCCTGAGCAAATATCTGAG |
| | | CGATCTGCTGTTTGTTTTTGGTCCGAATCTGCAGGATCC |
| | | GGTTAGCAATAGCATGACCATTCAGGCAATTAGCCAGG |
| | | CATTTGGTGGTAATTATGAAACCCTGCTGCGTACCCTGG |
| | | GTTATGCAACCGAAGATTTTGATGATCTGCTGGAAAGC |
| | | GATAGCATTACCGGTCAGATTATCTATGTTGATCTGAGC |
| | | AGCTACTATATTATCGTGCGTGTGTATTTTCCGATCCTG |
| | | ACCGAAATTCAGCAGGCATATATCCAAGAACTGCTGCC |
| | | GGTTAGCTTTAATAACGATAATAGCGAATGGATTAGCA |
| | | TCGTGCCGAACTTTATTCTGGTTCGTAATACCCTGATTA |
| | | GCAACATCGAAATTGGCTTTTGCCTGATTACCAAACGTA |
| | | GCGTGATTTGCAATCAGGATTATGCGACCCCGATGACC |
| | | AATAATATGCGTGAATGTCTGACAGGTAGCACCGAAAA |
| | | ATGTCCGCGTGAACTGGTTGTTAGCAGCCATGTTCCGCG |
| | | TTTTGCACTGAGCAATGGTGTTCTGTTTGCAAATTGTAT |
| | | TAGCGTTACCTGCCAGTGTCAGACCACCGGTCGTGCCAT |
| | | TAGCCAGAGCGGTGAACAGACCCTGCTGATGATTGATA |
| | | ATACCACCTGTCCGACCGCAGTTCTGGGTAATGTGATTA |
| | | TTAGCCTGGGTAAATATCTGGGCAGCGTGAATTATAAC |
| | | AGCGAAGGTATTGCCATTGGTCCGCCTGTTTTTACCGAT |
| | | AAAGTTGATATTAGCTCCCAGATCAGCAGCATGAATCA |
| | | GAGCCTGCAGCAGAGCAAAGATTATATCAAAGAAGCAC |
| | | AGCGTCTGCTGGATACCGTTAATCCGAGCCTGATTAGTA |
| | | TGCTGAGCATGATTATTCTGGGTAGCTCAGGAGCAAGT |
| | | CCGGCAGCACCGGCACCGGCATCACCAGCAGCTCCAGC |
| | | ACCTAGTGCACCGGCAGGCGGTATGGCAGAAATTGGCA |
| | | CCGGTTTTCCGTTTGATCCGCATTATGTTGAAGTTCTGG |
| | | GTGAACGTATGCATTATGTGGATGTTGGTCCGCGTGATG |
| | | GTACACCGGTTCTGTTTCTGCATGGTAATCCGACCAGCA |
| | | GCTATGTTTGGCGTAACATTATTCCGCATGTTGCACCGA |
| | | CACATCGTTGTATTGCACCGGATCTGATTGGTATGGGTA |
| | | AAAGCGATAAACCTGATCTGGGCTATTTCTTTGATGATC |
| | | ATGTGCGTTTTATGGACGCCTTTATTGAAGCACTGGGTT |
| | | TAGAAGAAGTTGTGCTGGTTATTCATGATTGGGGTAGT |
| | | GCCCTGGGTTTTCATTGGGCAAAACGTAATCCGGAACG |
| | | TGTTAAAGGTATTGCCTTTATGGAATTCATTCGTCCGAT |
| | | TCCGACCTGGGATGAATGGCCTGAATTTGCACGTGAAA |
| | | CCTTTCAGGCATTTCGTACCACCGATGTGGGTCGTAAAC |
| | | TGATTATTGATCAGAACGTTTTTATCGAAGGCACCCTGC |
| | | CGATGGGTGTTGTTCGTCCGCTGACCGAAGTTGAAATG |
| | | GATCATTATCGTGAACCGTTTCTGAATCCGGTTGATCGC |
| | | GAACCGCTGTGGCGTTTTCCGAATGAACTGCCGATTGCC |
| | | GGTGAACCTGCAAATATTGTTGCACTGGTTGAAGAGTA |
| | | TATGGATTGGCTGCATCAGAGTCCGGTTCCGAAACTGCT |
| | | GTTTTGGGGCACACCGGGTGTTCTGATTCCGCCTGCAGA |
| | | AGCAGCACGTCTGGCAAAAAGCCTGCCGAATTGTAAAG |
| | | CAGTTGATATTGGTCCGGGTCTGAATCTGCTGCAAGAA |
| | | GATAATCCAGATCTGATCGGTAGCGAAATTGCACGTTG |
| | | GCTGAGCACCCTGGAAATTAGCGGTCACATGGGTAGCG |
| | | AACAGAAACTGATCAGCGAAGAAGATCTGTAA |
| NIV G SNAP- | 13 | ATGGACTATAAAGACGATGACGACAAACACATGGGTAG |
| tag | | CATGGACAAAGATTGCGAAATGAAACGTACCACCCTGG |
| | | ATAGCCCGCTGGGCAAACTGGAACTGAGCGGCTGCGAA |
| | | CAGGGCCTGCATGAAATTAAACTGCTGGGTAAAGGCAC |
| | | CAGCGCGGCCGATGCGGTTGAAGTTCCGGCCCCGGCCG |
| | | CCGTGCTGGGTGGTCCGGAACCGCTGATGCAGGCGACC |
| | | GCGTGGCTGAACGCGTATTTTCATCAGCCGGAAGCGAT |
| | | TGAAGAATTTCCGGTTCCGGCGCTGCATCATCCGGTGTT |
| | | TCAGCAGGAGAGCTTTACCCGTCAGGTGCTGTGGAAAC |
| | | TGCTGAAAGTGGTTAAATTTGGCGAAGTGATTAGCTAT |
| | | CAGCAGCTGGCGGCCCTGGCGGGTAATCCGGCGGCCAC |
| | | CGCCGCCGTTAAAACCGCGCTGAGCGGTAACCCGGTGC |
| | | CGATTCTGATTCCGTGCCATCGTGTGGTTAGCTCTAGCG |
| | | GTGCGGTTGGCGGTTATGAAGGTGGTCTGGCGGTGAAA |
| | | GAGTGGCTGCTGGCCCATGAAGGTCATCGTCTGGGTAA |
| | | ACCGGGTCTGGGAGGTAGCTCAGGAGCAAGTCCGGCAG |
| | | CACCGGCACCGGCATCACCAGCAGCTCCAGCACCTAGT |
| | | GCACCGGCAGGCGGTCAAAATTACACCCGCTCGACAGA |
| | | TAACCAGGCAGTAATCAAGGACGCGTTACAAGGTATTC |
| | | AACAACAAATCAAAGGTCTTGCCGACAAGATTGGCACC |
| | | GAAATCGGTCCAAAGGTCAGCCTCATTGACACATCGAG |
| | | TACCATAACCATCCCAGCTAACATCGGCCTTCTGGGCTC |
| | | TAAAATTTCCCAGAGTACAGCCTCTATCAACGAGAACG |
| | | TAAACGAGAAATGCAAGTTTACTTTGCCCCCGCTGAAA |
| | | ATTCACGAATGCAACATTAGTTGTCCGAACCCTTTGCCA |
| | | TTTCGAGAGTACCGCCCCCAAACAGAGGGCGTGTCAAA |
| | | CTTGGTAGGACTCCCCAATAATATATGTCTCCAGAAGA |
| | | CTTCAAATCAAATACTGAAGCCTAAGTTGATATCATAC |
| | | ACCCTGCCGGTCGTCGGCCAAAGCGGCACATGTATAAC |
| | | AGACCCGCTCCTTGCTATGGACGAGGGTTATTTTGCCTA |
| | | CAGCCACCTGGAGCGCATCGGGTCATGTAGCCGTGGCG |
| | | TCTCTAAGCAGCGTATTATTGGCGTTGGGGAAGTCTTAG |
| | | ACCGTGGAGACGAGGTTCCTAGTTTATTCATGACCAAT |
| | | GTGTGGACACCCCCAAACCCCAATACGGTCTATCACTG |
| | | TTCTGCAGTATACAACAACGAATTTTACTACGTGCTTTG |
| | | TGCAGTATCAACCGTAGGCGATCCGATTCTCAACTCCAC |
| | | GTATTGGAGTGGAAGCTTGATGATGACGAGACTTGCTG |
| | | TTAAGCCAAAGTCGAATGGCGGTGGCTACAATCAACAT |
| | | CAATTAGCGCTCCGGAGTATTGAGAAGGGTCGCTACGA |
| | | CAAAGTTATGCCCTACGGCCCTAGTGGTATAAAGCAAG |
| | | GGGACACACTGTACTTCCCTGCAGTTGGTTTTCTGGTGC |
| | | GTACCGAATTCAAATACAACGACTCGAACTGCCCGATA |
| | | ACTAAATGCCAGTACTCCAAGCCTGAAAATTGCCGCCT |
| | | CTCGATGGGCATACGCCCTAATTCTCACTACATTTTGAG |
| | | ATCAGGGCTGCTTAAGTACAATCTGAGTGACGGCGAAA |
| | | ACCCCAAGGTGGTGTTTATCGAGATATCTGACCAAAGA |
| | | CTCTCCATCGGTAGCCCTTCCAAAATCTATGACTCACTT |
| | | GGTCAGCCCGTCTTCTATCAAGCTTCATTCAGTTGGGAC |
| | | ACAATGATTAAGTTCGGAGACGTTCTGACGGTAAACCC |
| | | TCTGGTCGTGAACTGGCGCAATAACACCGTTATAAGCC |
| | | GTCCAGGACAATCGCAGTGTCCTCGTTTCAACACCTGCC |
| | | CAGAGATCTGCTGGGAAGGCGTCTACAACGACGCGTTT |
| | | CTGATCGATCGGATTAACTGGATAAGTGCAGGGGTTTT |
| | | CTTGGACAGCAATCAAACCGCCGAAAACCCTGTTTTCA |
| | | CAGTGTTCAAGGACAACGAAATATTGTACCGCGCCCAA |
| | | CTTGCTTCTGAGGACACGAACGCACAGAAAACCATCAC |
| | | AAATTGCTTTCTGTTGAAGAATAAGATTTGGTGTATAAG |
| | | CCTTGTGGAAATCTACGACACTGGTGACAACGTCATAA |
| | | GACCGAAGTTATTTGCAGTGAAGATCCCGGAGCAATGC |
| | | ACAGAACAGAAACTGATCAGCGAAGAAGATCTGTAA |
| NiV G HaloTag | 14 | ATGGACTATAAAGACGATGACGACAAACACATGGGTAG |
| | | CATGGCAGAAATTGGCACCGGTTTTCCGTTTGATCCGCA |
| | | TTATGTTGAAGTTCTGGGTGAACGTATGCATTATGTGGA |
| | | TGTTGGTCCGCGTGATGGTACACCGGTTCTGTTTCTGCA |
| | | TGGTAATCCGACCAGCAGCTATGTTTGGCGTAACATTAT |
| | | TCCGCATGTTGCACCGACACATCGTTGTATTGCACCGGA |
| | | TCTGATTGGTATGGGTAAAAGCGATAAACCTGATCTGG |
| | | GCTATTTCTTTGATGATCATGTGCGTTTTATGGACGCCT |
| | | TTATTGAAGCACTGGGTTTAGAAGAAGTTGTGCTGGTTA |
| | | TTCATGATTGGGGTAGTGCCCTGGGTTTTCATTGGGCAA |
| | | AACGTAATCCGGAACGTGTTAAAGGTATTGCCTTTATG |
| | | GAATTCATTCGTCCGATTCCGACCTGGGATGAATGGCCT |
| | | GAATTTGCACGTGAAACCTTTCAGGCATTTCGTACCACC |
| | | GATGTGGGTCGTAAACTGATTATTGATCAGAACGTTTTT |
| | | ATCGAAGGCACCCTGCCGATGGGTGTTGTTCGTCCGCTG |
| | | ACCGAAGTTGAAATGGATCATTATCGTGAACCGTTTCTG |
| | | AATCCGGTTGATCGCGAACCGCTGTGGCGTTTTCCGAAT |
| | | GAACTGCCGATTGCCGGTGAACCTGCAAATATTGTTGC |
| | | ACTGGTTGAAGAGTATATGGATTGGCTGCATCAGAGTC |
| | | CGGTTCCGAAACTGCTGTTTTGGGGCACACCGGGTGTTC |
| | | TGATTCCGCCTGCAGAAGCAGCACGTCTGGCAAAAAGC |
| | | CTGCCGAATTGTAAAGCAGTTGATATTGGTCCGGGTCTG |
| | | AATCTGCTGCAAGAAGATAATCCAGATCTGATCGGTAG |
| | | CGAAATTGCACGTTGGCTGAGCACCCTGGAAATTAGCG |
| | | GTGGTAGCTCAGGAGCAAGTCCGGCAGCACCGGCACCG |
| | | GCATCACCAGCAGCTCCAGCACCTAGTGCACCGGCAGG |
| | | CGGTCAAAATTACACCCGCTCGACAGATAACCAGGCAG |
| | | TAATCAAGGACGCGTTACAAGGTATTCAACAACAAATC |
| | | AAAGGTCTTGCCGACAAGATTGGCACCGAAATCGGTCC |
| | | AAAGGTCAGCCTCATTGACACATCGAGTACCATAACCA |
| | | TCCCAGCTAACATCGGCCTTCTGGGCTCTAAAATTTCCC |
| | | AGAGTACAGCCTCTATCAACGAGAACGTAAACGAGAAA |
| | | TGCAAGTTTACTTTGCCCCCGCTGAAAATTCACGAATGC |
| | | AACATTAGTTGTCCGAACCCTTTGCCATTTCGAGAGTAC |
| | | CGCCCCCAAACAGAGGGCGTGTCAAACTTGGTAGGACT |
| | | CCCCAATAATATATGTCTCCAGAAGACTTCAAATCAAA |
| | | TACTGAAGCCTAAGTTGATATCATACACCCTGCCGGTCG |
| | | TCGGCCAAAGCGGCACATGTATAACAGACCCGCTCCTT |
| | | GCTATGGACGAGGGTTATTTTGCCTACAGCCACCTGGA |
| | | GCGCATCGGGTCATGTAGCCGTGGCGTCTCTAAGCAGC |
| | | GTATTATTGGCGTTGGGGAAGTCTTAGACCGTGGAGAC |
| | | GAGGTTCCTAGTTTATTCATGACCAATGTGTGGACACCC |
| | | CCAAACCCCAATACGGTCTATCACTGTTCTGCAGTATAC |
| | | AACAACGAATTTTACTACGTGCTTTGTGCAGTATCAACC |
| | | GTAGGCGATCCGATTCTCAACTCCACGTATTGGAGTGG |
| | | AAGCTTGATGATGACGAGACTTGCTGTTAAGCCAAAGT |
| | | CGAATGGCGGTGGCTACAATCAACATCAATTAGCGCTC |
| | | CGGAGTATTGAGAAGGGTCGCTACGACAAAGTTATGCC |
| | | CTACGGCCCTAGTGGTATAAAGCAAGGGGACACACTGT |
| | | ACTTCCCTGCAGTTGGTTTTCTGGTGCGTACCGAATTCA |
| | | AATACAACGACTCGAACTGCCCGATAACTAAATGCCAG |
| | | TACTCCAAGCCTGAAAATTGCCGCCTCTCGATGGGCAT |
| | | ACGCCCTAATTCTCACTACATTTTGAGATCAGGGCTGCT |
| | | TAAGTACAATCTGAGTGACGGCGAAAACCCCAAGGTGG |
| | | TGTTTATCGAGATATCTGACCAAAGACTCTCCATCGGTA |
| | | GCCCTTCCAAAATCTATGACTCACTTGGTCAGCCCGTCT |
| | | TCTATCAAGCTTCATTCAGTTGGGACACAATGATTAAGT |
| | | TCGGAGACGTTCTGACGGTAAACCCTCTGGTCGTGAAC |
| | | TGGCGCAATAACACCGTTATAAGCCGTCCAGGACAATC |
| | | GCAGTGTCCTCGTTTCAACACCTGCCCAGAGATCTGCTG |
| | | GGAAGGCGTCTACAACGACGCGTTTCTGATCGATCGGA |
| | | TTAACTGGATAAGTGCAGGGGTTTTCTTGGACAGCAAT |
| | | CAAACCGCCGAAAACCCTGTTTTCACAGTGTTCAAGGA |
| | | CAACGAAATATTGTACCGCGCCCAACTTGCTTCTGAGG |
| | | ACACGAACGCACAGAAAACCATCACAAATTGCTTTCTG |
| | | TTGAAGAATAAGATTTGGTGTATAAGCCTTGTGGAAAT |
| | | CTACGACACTGGTGACAACGTCATAAGACCGAAGTTAT |
| | | TTGCAGTGAAGATCCCGGAGCAATGCACAGAACAGAAA |
| | | CTGATCAGCGAAGAAGATCTGTAA |

1. A vaccine composition comprising:
a nanocarrier comprising a membrane;
a cell-free protein synthesis system; and
at least one of a nucleic acid template encoding a NiVG protein comprising a transmembrane domain and a nucleic acid template encoding a NiVF protein comprising a transmembrane domain;
wherein the NiVG transmembrane domain and the NiVF transmembrane domain are able to integrate into the nanocarrier membrane.

2. The composition of claim 1, wherein the composition comprises the nucleic acid template encoding the NiVG protein and the nucleic acid template encoding the NiVF protein.

3. The composition of claim 1 or claim 2, wherein the NiVF protein comprises SEQ ID NO: 1 or a sequence having at least 90% identity thereto, and wherein the NiVG protein comprises SEQ ID NO: 3 or a sequence having at least 90% identity thereto.

4. The composition of claim 1 or claim 2, wherein the NiVF protein comprises a deletion of the signal peptide sequence.

5. The composition of claim 4, wherein the NiVF protein comprises SEQ ID NO: 2 or a sequence having at least 90% identity thereto.

6. The composition of any one of claims 1-5, wherein the nanocarrier is a liposome, a polymersome, or a lipid nanoparticle.

7. The composition of claim 6, wherein the nanocarrier is a liposome.

8. The composition of claim 7, wherein the liposome membrane comprises phosphatidylcholine (PC) headgroups.

9. The composition of claim 8, wherein the liposome membrane comprises a mixture of PC headgroups and phosphoethanolamine (PE) headgroups; optionally wherein the PC and PE headgroups are comprised at a ratio of about 4:1 PC:PE.

10. The composition of claim 8, wherein the liposome membrane comprises a mixture of PC headgroups, PE headgroups, and phosphatidylserine (PS) headgroups; optionally wherein the PC, PE, and PS headgroups are comprised at a ratio of about 15:4:1 PC:PE:PS.

11. The composition of any one of claims 8-10, wherein the liposome membrane further comprises a lipid adjuvant; optionally wherein the lipid adjuvant is monophosphoryl lipid A (MPLA) at a concentration of between about 0.004% and about 0.4%.

12. The composition of any one of claims 1-11, wherein the nanocarrier further comprises a cargo molecule.

13. The composition of claim 12, wherein the cargo molecule comprises a polynucleotide, a polypeptide, an active pharmaceutical ingredient, or a therapeutic agent.

14. A vaccine nanoparticle prepared from the composition of any one of claims 1-13, wherein the vaccine nanoparticle is prepared by expressing the nucleic acid template encoding the NiVG protein or the nucleic acid template encoding the NiVF protein; optionally wherein the method comprises co-expressing the nucleic acid template encoding the NiVG protein and the nucleic acid template encoding the NiVF protein.

15. A vaccine nanoparticle comprising the nanocarrier and at least one of the NiVG protein or the NiVF fusion protein according to any one of claims 1-11, wherein at least one of the NiVG transmembrane domain and the NiVF transmembrane domain is integrated into the nanocarrier membrane.

16. The vaccine nanoparticle of claim 15, wherein the vaccine nanoparticle comprises both the NiVG protein and the NiVF protein and wherein the NivG transmembrane domain and the NiVF transmembrane domain are integrated into the nanocarrier membrane.

17. A vaccine composition comprising:
a nanocarrier comprising a membrane;
a cell-free protein synthesis system; and
at least one of a nucleic acid template encoding a NiVG fusion protein and a nucleic acid template encoding a NiVF fusion protein,
wherein the membrane comprises at least one of a first modified lipid and a second modified lipid; and
wherein the NiVG fusion protein comprises a first protein tag that conjugates to the first modified lipid and a NiVG Nipah virus protein, and
wherein the NiVF fusion protein comprises a second protein tag that conjugates to the second modified lipid and a NiVF Nipah virus protein.

18. The composition of claim 17, wherein the composition comprises the nucleic acid template encoding the NiVG fusion protein and the nucleic acid template encoding the NiVF fusion protein.

19. The composition of claim 17 or claim 18, wherein the NiVF Nipah virus protein comprises SEQ ID NO: 1 or a sequence having at least 90% identity thereto, and wherein the NiVG Nipah virus protein comprises SEQ ID NO: 3 or a sequence having at least 90% identity thereto.

20. The composition of claim 17 or claim 18, wherein the NiVF protein comprises a deletion of the signal peptide sequence.

21. The composition of claim 20, wherein the NiVF protein comprises SEQ ID NO: 2 or a sequence having at least 90% identity thereto.

22. The composition of any one of claims 17-21, wherein the first modified lipid and the second modified lipid are the same.

23. The composition of any one of claims 17-21, wherein the first modified lipid and the second modified lipid are different.

24. The composition of any one of claims 17-21, wherein the first protein tag and the second protein tag are self-labeling enzymes.

25. The composition of claim 24, wherein at least one of the first and second modified lipid comprises benzylguanine and at least one of the self-labeling enzyme is a SNAP-tag.

26. The composition of claim 24, wherein at least one of the first and second modified lipid comprises a HaloTag ligand and at least one of the first and second self-labeling enzyme is a HaloTag.

27. The composition of any one of claims 17-26, wherein the nanocarrier is a liposome, a polymersome, or a lipid nanoparticle.

28. The composition of claim 27, wherein the nanocarrier is a liposome.

29. The composition of claim 28, wherein the liposome membrane comprises phosphatidylcholine (PC) headgroups.

30. The composition of claim 29, wherein the liposome membrane comprises a mixture of PC headgroups and phosphoethanolamine (PE) headgroups; optionally wherein the PC and PE headgroups are comprised at a ratio of about 4:1 PC:PE.

31. The composition of claim 29, wherein the liposome membrane comprises a mixture of PC headgroups, PE headgroups, and phosphatidylserine (PS) headgroups; optionally wherein the PC, PE, and PS headgroups are comprised at a ratio of about 15:4:1 PC:PE:PS.

32. The composition of any one of claims 29-31, wherein the liposome membrane further comprises a lipid adjuvant; optionally wherein the lipid adjuvant is monophosphoryl lipid A (MPLA) at a concentration of between about 0.004% and about 0.4%.

33. The composition of any one of claims 17-32, wherein the nanocarrier further comprises a cargo molecule.

34. The composition of claim 33, wherein the cargo molecule comprises a polynucleotide, a polypeptide, an active pharmaceutical ingredient, or a therapeutic agent.

35. A vaccine nanoparticle prepared from the composition according to any one of claims 17-34, wherein the vaccine nanoparticle is prepared by expressing the nucleic acid template encoding the NiVG fusion protein or the nucleic acid template encoding the NiVF fusion protein; optionally wherein the method comprises co-expressing the nucleic acid template encoding the NiVG fusion protein and the nucleic acid template encoding the NiVF fusion protein.

36. A vaccine nanoparticle comprising the nanocarrier and at least one of the NiVG fusion protein and the NiVF fusion protein according to any one of claims 17-34, wherein the at least one protein tag is associated with the modified lipid.

37. The vaccine nanoparticle of claim 36, wherein the vaccine nanoparticle comprises both the NiVG fusion protein and the NiVF fusion protein and wherein the first and second protein tags are associated with the first and second modified lipid.

38. A method for eliciting neutralizing antibodies in a subject, the method comprising administering the vaccine nanoparticle of any one of claim 14-16 or 35-37 to the subject.

39. A method for treating a Nipah virus infection in a subject, the method comprising administering to the subject a therapeutically effective amount of the vaccine nanoparticle of any one of claim 14-16 or 35-37.

40. A method for preparing a vaccine nanoparticle, the method comprising expressing at least one of the nucleic acid template encoding the NiVG protein and the nucleic acid template encoding the NiVF protein in the composition according to any one of claims 1-13.

41. The method of claim 34, wherein the method comprises co-expressing the nucleic acid template encoding the NiVG protein and the nucleic acid template encoding the NiVF protein.

42. A method for preparing a vaccine nanoparticle, the method comprising expressing at least one of the nucleic acid template encoding the NiVG fusion protein and the nucleic acid template encoding the NiVF fusion protein in the composition according to any one of claims 17-34.

43. The method of claim 42, wherein the method comprises co-expressing the nucleic acid template encoding the NiVG fusion protein and the nucleic acid template encoding the NiVF fusion protein.
